(19) United States
(12) Patent Application Publication
(10) Pub. No.: US 2024/0207269 A1
(43) Pub. Date: Jun. 27, 2024

(54) USES OF HETEROCYCLIC INHIBITORS OF ERK1/2

(71) Applicant: Erasca, Inc., San Diego, CA (US)

(72) Inventors: Dawei XUAN, San Diego, CA (US); Xiao Qing YANG, San Diego, CA (US); Wei LIN, San Diego, CA (US)

(21) Appl. No.: 18/555,071

(22) PCT Filed: Apr. 14, 2022
(86) PCT No.: PCT/US2022/024841

§ 371 Date: Oct. 12, 2023

Related U.S. Application Data
(60) Provisional application No. 63/176,131, filed on Apr. 16, 2021.

(51) Int. Cl. A61K 31/506; A61P 35/00

(52) U.S. Cl. CPC A61K 31/506; A61P 35/00

(57) ABSTRACT

The use of heterocyclic inhibitors of ERK1/2 for treating cancer, specifically solid tumors. In particular, the ERK1/2 N inhibitor is (S)—N-(2-amino-1-(3-chloro-5-fluorophenyl)ethyl)-1-(5-methyl-2-((tetrahydro2H-pyran-4-yl)amino)pyrimidin-4-yl)-1H-imidazole-4-carboxamide which is administered to a subject twice a day, once a week and is used to treat cancers including non-small cell lung cancer (NSCLC), melanoma, pancreatic cancer, or colorectal cancer (CRC). In some embodiments, the cancer is a MAP-Km/MAPKi-naive pan tumor.

### CROSS-REFERENCE

This application claims the benefit of U.S. Provisional Application Ser. No. 63/176,131 filed Apr. 16, 2021 which is hereby incorporated by reference in its entirety.

### BACKGROUND OF THE INVENTION

ERK1 and ERK2 (collectively “ERK1/2”) are related protein-serine/threonine kinases that participate in, amongst others, the Ras-Raf-MEK-ERK signal transduction pathway, which is sometimes denoted as the mitogen-activated protein kinase (MAPK) pathway. This pathway is thought to play a central role in regulating a number of fundamental cellular processes including one or more of cell proliferation, survival, adhesion, cycle progression, migration, differentiation, metabolism, and transcription. The activation of the MAPK pathway has been reported in numerous tumor types including lung, colon, pancreatic, renal, and ovarian cancers. Accordingly, substances that could reduce activation could be of interest for possible treatments.

ERK1/2 appear to be activated by MEK through phosphorylation of both a threonine and a tyrosine residue, namely at Tyr204/187 and Thr202/185. Once activated, ERK1/2 catalyze the phosphorylation of serine/threonine residues of more than 100 substrates and activate both cytosolic and nuclear proteins that are linked to cell growth, proliferation, survival, angiogenesis and differentiation, all hallmarks of the cancer phenotype. Thus it may be beneficial to target ERK 1 and ERK 2 to develop and use ERK1/2 inhibitors as a way to inhibit tumor growth.

Furthermore, an ERK inhibitor may have utility in combination with other kinase, for example MAPK, inhibitors. Recently, researchers reported that dual inhibition of MEK and ERK by small molecule inhibitors was synergistic and acted to overcome acquired resistance to MEK inhibitors. See Hatzivassiliou et al., ERK Inhibition Overcomes Acquired Resistance to MEK Inhibition, Mol. Cancer Ther. 2012, 11, 1143-1154.

There is a need to develop dosing regimens that improve exposure, prolong ERK1/2 inhibition, and increase antitumor activity while maintaining an acceptable safety profile.

### SUMMARY OF THE INVENTION

In a first aspect, the present disclosure provides a method of treating cancer in a subject in need thereof, the method comprising administering to the subject in need thereof:
compound 1

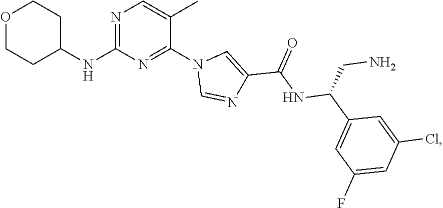 [SMILES: Cc1cnc(NC2CCOCC2)nc1-n1cnc(C(=O)N[C@H](CN)c2cc(F)cc(Cl)c2)c1]

or a pharmaceutically acceptable salt thereof, wherein compound 1 is administered twice a day, once a week (BID-QW).

In some embodiments of a method of treating cancer, the salt of compound 1, is the mandelic acid salt.

In some embodiments of a method of treating cancer, compound 1, or a pharmaceutically acceptable salt thereof, is administered for at least one 28-day cycle.

In some embodiments of a method of treating cancer, compound 1, or a pharmaceutically acceptable salt thereof, is administered twice a day on day 1, day 8, day 15, and day 22 of a 28-day cycle.

In some embodiments of a method of treating cancer, compound 1, or a pharmaceutically acceptable salt thereof, is administered twice a day on day 1, day 8, day 15 of a 28-day cycle.

In some embodiments of a method of treating cancer, compound 1, or a pharmaceutically acceptable salt thereof, is not administered on days 2-7, days 9-14, days 16-21, and days 23-28 of a 28-day cycle.

In some embodiments of a method of treating cancer, compound 1, or a pharmaceutically acceptable salt thereof, is administered for at least one 35-day cycle.

In some embodiments of a method of treating cancer, compound 1, or a pharmaceutically acceptable salt thereof, is administered twice a day on day 1, day 8, day 15, and day 22 of a 35-day cycle.

In some embodiments of a method of treating cancer, compound 1, or a pharmaceutically acceptable salt thereof, is not administered on days 2-7, days 9-14, days 16-21, days 23-28, and days 30-35 of a 28-day cycle.

In some embodiments of a method of treating cancer, compound 1, or a pharmaceutically acceptable salt thereof, is administered in an amount that is between about 25 mg and about 300 mg twice a day, once a week (BID-QW).

In some embodiments of a method of treating cancer, compound 1, or a pharmaceutically acceptable salt thereof, is administered in an amount that is between about 25 mg and about 250 mg twice a day, once a week (BID-QW).

In some embodiments of a method of treating cancer, compound 1, or a pharmaceutically acceptable salt thereof, is administered in an amount that is between about 25 mg and about 150 mg twice a day, once a week (BID-QW).

In some embodiments of a method of treating cancer, compound 1, or a pharmaceutically acceptable salt thereof, is administered in an amount that is about 25 mg, 50 mg, about 75 mg, about 100 mg, about 125 mg, about 150 mg, about 175 mg, about 200 mg, about 225 mg, or about 250 mg twice a day, once a week (BID-QW).

In some embodiments of a method of treating cancer, compound 1, or a pharmaceutically acceptable salt thereof, is administered in an amount that is about 25 mg, 50 mg, about 100 mg, about 125 mg, or about 150 mg twice a day, once a week (BID-QW).

In some embodiments of a method of treating cancer, the cancer comprises at least one cancer cell with one or more activating mutations in the MAPK pathway.

In some embodiments of a method of treating cancer, the cancer is a mitogen-activated protein kinase (MAPK) pathway driven cancer.

In some embodiments of a method of treating cancer, the cancer is a BRAF-driven cancer, HRAS-driven cancer, or a NRAS-driven cancer.

In some embodiments of a method of treating cancer, the cancer comprises at least one cancer cell driven by deregulated ERK.

In some embodiments of a method of treating cancer, the cancer has at least one mutation in RAS, RAF, or MEK.

In some embodiments of a method of treating cancer, the subject has not previously received a MAPK pathway inhibitor (e.g., a KRAS inhibitor, a BRAF inhibitor, a MEK inhibitor, or an ERK inhibitor).

In some embodiments of a method of treating cancer, the cancer is colorectal cancer (CRC), pancreatic ductal adenocarcinoma (PDAC), cholangiocarcinoma cancer, appendiceal cancer, gastric cancer, esophageal cancer, non-small cell lung cancer (NSCLC), head and neck cancer, ovarian cancer, uterine cancer, or acute myeloid leukemia (AML).

In some embodiments of a method of treating cancer, the cancer is non-small cell lung cancer (NSCLC), melanoma, pancreatic cancer, or colorectal cancer (CRC).

In some embodiments of a method of treating cancer, the cancer is a MAPKm/MAPKi-naïve pan tumor, MAPKm/MAPKi-naïve pancreatic cancer, MAPKm/MAPKi-naïve NSCLC, BRAFi-treated V600 NSCLC, BRAFi-treated V600 melanoma, or KRAS-treated G12C NSCLC.

In some embodiments of a method of treating cancer, the subject in need thereof does not experience serious adverse events during the 28-day cycles.

In some embodiments of a method of treating cancer, the subject in need thereof does not experience Grade 3, 4, or 5 adverse events during the 28-day cycles.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIG. 1 shows a plot of simulated Compound 1 steady-state PK profiles.

### DETAILED DESCRIPTION OF THE INVENTION

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included.

The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

As used herein, the term “therapeutic” means an agent utilized to treat, combat, ameliorate, prevent or improve an unwanted condition or disease of a patient. In some embodiments, a therapeutic agent such as a compound 1 is directed to the treatment and/or the amelioration of cancers.

“Administering” when used in conjunction with a therapeutic means to administer a therapeutic systemically or locally, as directly into or onto a target tissue, or to administer a therapeutic to a patient whereby the therapeutic positively impacts the tissue to which it is targeted. Thus, as used herein, the term “administering”, when used in conjunction with a composition described herein, can include, but is not limited to, providing a composition into or onto the target tissue; providing a composition systemically to a patient by, e.g., oral administration whereby the therapeutic reaches the target tissue or cells. “Administering” a composition may be accomplished by injection, topical administration, and oral administration or by other methods alone or in combination with other known techniques.

The term “animal” as used herein includes, but is not limited to, humans and non-human vertebrates such as wild, domestic and farm animals. As used herein, the terms “patient,” “subject” and “individual” are intended to include living organisms in which certain conditions as described herein can occur. Examples include humans, monkeys, cows, sheep, goats, dogs, cats, mice, rats, and transgenic species thereof. In a preferred embodiment, the patient is a primate. In certain embodiments, the primate or subject is a human. In certain instances, the human is an adult. In certain instances, the human is child. In further instances, the human is under the age of 12 years. In certain instances, the human is elderly. In other instances, the human is 60 years of age or older. Other examples of subjects include experimental animals such as mice, rats, dogs, cats, goats, sheep, pigs, and cows. The experimental animal can be an animal model for a disorder, e.g., a transgenic mouse with hypertensive pathology.

By “pharmaceutically acceptable”, it is meant the carrier, diluent or excipient must be compatible with the other ingredients of the formulation and not deleterious to the recipient thereof.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

A “therapeutically effective amount” or “effective amount” as used herein refers to the amount of active compound or pharmaceutical agent that elicits a biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease, (2) inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) ameliorating the disease; for example, ameliorating a disease, condition or disorder in an individual that is experiencing or displaying the pathology or symptomatology of the disease, condition or disorder (i.e., reversing the pathology and/or symptomatology).

The terms “treat,” “treated,” “treatment,” or “treating” as used herein refers to both therapeutic treatment in some embodiments and prophylactic or preventative measures in other embodiments, wherein the object is to prevent or slow (lessen) an undesired physiological condition, disorder or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. A prophylactic benefit of treatment includes prevention of a condition, retarding the progress of a condition, stabilization of a condition, or decreasing the likelihood of occurrence of a condition. As used herein, “treat,” “treated,” “treatment,” or “treating” includes prophylaxis in some embodiments.

The term “substantially the same as” as used herein, refers to a powder x-ray diffraction pattern or differential scanning calorimetry pattern that is non-identical to those depicted herein, but that falls within the limits of experimental error, when considered by one of ordinary skill in the art.

### Compound 1

Disclosed herein is (S)—N-(2-amino-1-(3-chloro-5-fluorophenyl)ethyl)-1-(5-methyl-2-((tetrahydro-2H-pyran-4-yl)amino)pyrimidin-4-yl)-1H-imidazole-4-carboxamide:

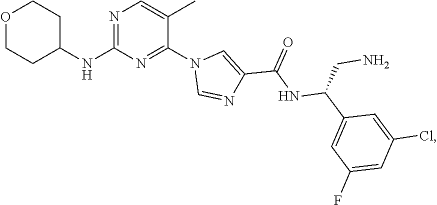 [SMILES: Cc1cnc(NC2CCOCC2)nc1-n1cnc(C(=O)N[C@H](CN)c2cc(F)cc(Cl)c2)c1]

or a pharmaceutically acceptable salt thereof.

In some embodiments, the salt of compound 1 is the mandelic acid salt. In some embodiments, the salt of compound 1 is the benzenesulfonic acid salt. In some embodiments, the salt of compound 1 is the hydrochloride salt. In some embodiments, the salt of compound 1 is the p-toluenesulfonic acid salt.

In some embodiments, the salt of compound 1 is the benzenesulfonic acid salt.

### Methods of Treatment

Disclosed herein is a method of treating cancer, in a subject in need thereof; the method comprising administering a compound 1, or a pharmaceutically acceptable salt thereof, wherein compound 1 is administered twice a day, once weekly (BID-QW). Also disclosed herein is a method of treating a solid tumor, in a subject in need thereof; the method comprising administering a compound 1, or a pharmaceutically acceptable salt thereof, wherein compound 1 is administered twice a day, once weekly (BID-QW). In some embodiments, compound 1 is administered for at least one 28-day cycle. In some embodiments, compound 1 is administered for at least one 35-day cycle.

In some embodiments, the cancer is non-small cell lung cancer (NSCLC), melanoma, pancreatic cancer (such as pancreatic cancer is pancreatic ductal adenocarcinoma, PDAC), salivary gland tumor, thyroid cancer, colorectal cancer (CRC), or esophageal cancer.

In some embodiments, the cancer is colorectal cancer (CRC), pancreatic ductal adenocarcinoma (PDAC), cholangiocarcinoma cancer, appendiceal cancer, gastric cancer, esophageal cancer, non-small cell lung cancer (NSCLC), head and neck cancer, ovarian cancer, or uterine cancer.

In some embodiments, the cancer is a liquid tumor, hematologic malignancy, or a blood cancer. In some embodiments, the cancer is a leukemia, lymphoma, or melanoma. In some embodiments, the cancer is acute myeloid leukemia (AML). In some embodiments, AML is relapsed and/or refractory AML. In some embodiments, AML is a FLT3 mutant AML.

In some embodiments, the cancer is a solid tumor.

In some embodiments, the solid tumor is an advanced or a metastatic solid tumor. In some embodiments, the solid tumor is non-small cell lung cancer (NSCLC), melanoma, pancreatic cancer, or colorectal cancer (CRC).

In some embodiments of a method of treating a solid tumor, the tumor is metastatic BRAF mutated melanoma; metastatic NRASmut or HRASmut advanced solid tumors; metastatic KRASmut colorectal cancer (CRC); metastatic KRASmut colorectal cancer (CRC); metastatic KRASmut non-small cell lung cancer (NSCLC); metastatic pancreatic ductal adenocarcinoma (PDAC).

In some embodiments of a method of treating a solid tumor, the solid tumor is a NSCLC, wherein the NSCLC is a MAPKm/MAPKi-naïve NSCLC. In some embodiments of a method of treating a solid tumor, the solid tumor is a NSCLC, wherein the NSCLC is a BRAFi-treated V600 NSCLC. In some embodiments of a method of treating a solid tumor, the solid tumor is a NSCLC, wherein the NSCLC is a KRAS-treated G12C NSCLC.

In some embodiments of a method of treating a solid tumor, the solid tumor is a pancreatic cancer, wherein the pancreatic cancer is a MAPKm/MAPKi-naïve pancreatic cancer.

In some embodiments of a method of treating a solid tumor, the solid tumor is a MAPKm/MAPKi-naïve pan tumor.

In some embodiments of a method of treating a solid tumor, the solid tumor comprises at least one cancer cell with one or more activating mutations in the MAPK pathway.

In some embodiments of a method of treating a solid tumor, the tumor is a mitogen-activated protein kinase (MAPK) pathway driven cancer. In some embodiments of a method of treating a solid tumor, the cancer is a BRAF-driven cancer. In some embodiments of a method of treating a solid tumor, the cancer is a HRAS-driven cancer. In some embodiments of a method of treating a solid tumor, the cancer is a NRAS-driven cancer.

### Dosing

In one aspect, the compositions described herein are used for the treatment of diseases and conditions described herein. In addition, a method for treating any of the diseases or conditions described herein in a subject in need of such treatment, involves administration of compositions in therapeutically effective amounts to said subject.

Dosages of compositions described herein can be determined by any suitable method. Maximum tolerated doses (MTD) and maximum response doses (MRD) for compound 1, or a pharmaceutically acceptable salt thereof can be determined via established animal and human experimental protocols as well as in the examples described herein. For example, toxicity and therapeutic efficacy of compound 1, or a pharmaceutically acceptable salt thereof, can be determined by standard pharmaceutical procedures in cell cultures or experimental animals, including, but not limited to, for determining the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized. Additional relative dosages, represented as a percent of maximal response or of maximum tolerated dose, are readily obtained via the protocols.

In some embodiments, the amount of a given formulation comprising compound 1, or a pharmaceutically acceptable salt thereof that corresponds to such an amount varies depending upon factors such as the molecular weight of a particular salt or form, disease condition and its severity, the identity (e.g., age, weight, sex) of the subject or host in need of treatment, but can nevertheless be determined according to the particular circumstances surrounding the case, including, e.g., the specific agent being administered, the liquid formulation type, the condition being treated, and the subject or host being treated.

In some embodiments, the amount of compound 1, or a pharmaceutically acceptable salt thereof, as described herein is relative to the free-base equivalent of compound 1.

In some embodiments, compound 1, or a pharmaceutically acceptable salt thereof, is administered in an amount between about 25 mg to about 300 mg twice a day, once a week (BID-QW).

In some embodiments, compound 1, or a pharmaceutically acceptable salt thereof, is administered in an amount that is between about 25 mg and about 250 mg twice a day, once a week (BID-QW).

In some embodiments, compound 1, or a pharmaceutically acceptable salt thereof, is administered in an amount that is between about 25 mg and about 200 twice a day, once a week (BID-QW).

In some embodiments, compound 1, or a pharmaceutically acceptable salt thereof, is administered in an amount that is between about 25 mg and about 150 mg twice a day, once a week (BID-QW).

In some embodiments, compound 1, or a pharmaceutically acceptable salt thereof, is administered in an amount that is between about 25 mg and about 100 mg twice a day, once a week (BID-QW).

In some embodiments, compound 1, or a pharmaceutically acceptable salt thereof, is administered in an amount that is between about 25 mg and about 50 mg twice a day, once a week (BID-QW).

In some embodiments, compound 1, or a pharmaceutically acceptable salt thereof, is administered in an amount that is between about 50 mg to about 300 mg twice a day, once a week (BID-QW).

In some embodiments, compound 1, or a pharmaceutically acceptable salt thereof, is administered in an amount that is between about 50 mg and about 250 twice a day, once a week (BID-QW).

In some embodiments, compound 1, or a pharmaceutically acceptable salt thereof, is administered in an amount that is between about 50 mg and about 200 mg twice a day, once a week (BID-QW).

In some embodiments, compound 1, or a pharmaceutically acceptable salt thereof, is administered in an amount that is between about 50 mg and about 150 mg twice a day, once a week (BID-QW).

In some embodiments, compound 1, or a pharmaceutically acceptable salt thereof, is administered in an amount that is between about 50 mg and about 100 mg twice a day, once a week (BID-QW).

In some embodiments, compound 1, or a pharmaceutically acceptable salt thereof, is administered in an amount that is between about 100 mg and about 300 mg twice a day, once a week (BID-QW).

In some embodiments, compound 1, or a pharmaceutically acceptable salt thereof, is administered in an amount that is between about 100 mg and about 250 mg twice a day, once a week (BID-QW).

In some embodiments, compound 1, or a pharmaceutically acceptable salt thereof, is administered in an amount that is between about 100 mg and about 200 mg twice a day, once a week (BID-QW).

In some embodiments, compound 1, or a pharmaceutically acceptable salt thereof, is administered in an amount that is between about 100 mg and about 150 mg twice a day, once a week (BID-QW).

In some embodiments, compound 1, or a pharmaceutically acceptable salt thereof, is administered in an amount that is between about 150 mg and about 300 mg twice a day, once a week (BID-QW).

In some embodiments, compound 1, or a pharmaceutically acceptable salt thereof, is administered in an amount that is between about 150 mg and about 250 mg twice a day, once a week (BID-QW).

In some embodiments, compound 1, or a pharmaceutically acceptable salt thereof, is administered in an amount that is between about 150 mg and about 200 mg twice a day, once a week (BID-QW).

In some embodiments, compound 1, or a pharmaceutically acceptable salt thereof, is administered in an amount that is between about 175 mg and about 300 mg twice a day, once a week (BID-QW).

In some embodiments, compound 1, or a pharmaceutically acceptable salt thereof, is administered in an amount that is between about 175 mg and about 250 mg twice a day, once a week (BID-QW).

In some embodiments, compound 1, or a pharmaceutically acceptable salt thereof, is administered in an amount that is between about 175 mg and about 200 mg twice a day, once a week (BID-QW).

In some embodiments, compound 1, or a pharmaceutically acceptable salt thereof, is administered in an amount that is between about 200 mg and about 300 mg twice a day, once a week (BID-QW).

In some embodiments, compound 1, or a pharmaceutically acceptable salt thereof, is administered in an amount that is between about 200 mg and about 250 mg twice a day, once a week (BID-QW).

In some embodiments, compound 1, or a pharmaceutically acceptable salt thereof, is administered in an amount that is between about 225 mg and about 300 mg twice a day, once a week (BID-QW).

In some embodiments, compound 1, or a pharmaceutically acceptable salt thereof, is administered in an amount that is between about 225 mg and about 250 mg twice a day, once a week (BID-QW).

In some embodiments, compound 1, or a pharmaceutically acceptable salt thereof, is administered in an amount that is about 25 mg, 30 mg, 40 mg, 50 mg, about 60 mg, about 70 mg, about 75 mg, about 80 mg, about 90 mg, about 100 mg, about 110 mg, about 120 mg, about 125 mg, about 130 mg, about 140 mg, about 150 mg, about 160 mg, about 170 mg, about 175 mg, about 180 mg, about 190 mg, about 200 mg, about 210 mg, about 220 mg, about 225 mg, about 230 mg, about 240 mg, about 250 mg, about 260 mg, about 270 mg, about 280 mg, about 290 mg, or about 300 mg.

### Administration

Administration of compound 1, or a pharmaceutically acceptable salt thereof, described is at a dosage described herein or at other dose levels and compositions determined and contemplated by a medical practitioner. In certain embodiments, compound 1, or a pharmaceutically acceptable salt thereof, is administered for prophylactic and/or therapeutic treatments. In certain therapeutic applications, compound 1, or a pharmaceutically acceptable salt thereof, is administered to a patient already suffering from a disease in an amount sufficient to cure the disease or at least partially arrest or ameliorate the symptoms. Amounts effective for this use depend on the age of the patient, severity of the disease, previous therapy, the patient's health status, weight, and response to the compositions, and the judgment of the treating physician. Therapeutically effective amounts are optionally determined by methods including, but not limited to, a dose escalation clinical trial.

In prophylactic applications, the compositions described herein are administered to a patient susceptible to or otherwise at risk of a particular disease, e.g., cancer. Such an amount is defined to be a “prophylactically effective amount or dose.” In this use, the precise amounts also depend on the patient's age, state of health, weight, and the like. When used in a patient, effective amounts for this use will depend on the risk or susceptibility of developing the particular disease, previous therapy, the patient's health status and response to the compositions, and the judgment of the treating physician.

In certain embodiments wherein the patient's condition does not improve, upon the doctor's discretion the administration of a composition described herein are administered chronically, that is, for an extended period of time, including throughout the duration of the patient's life in order to ameliorate or otherwise control or limit the symptoms of the patient's disease. In other embodiments, administration of a composition continues until complete or partial response of a disease.

In some embodiments, compound 1, or a pharmaceutically acceptable salt thereof, is administered once a day. In some embodiments, compound 1, or a pharmaceutically acceptable salt thereof, is administered twice a day. In some embodiments, compound 1, or a pharmaceutically acceptable salt thereof, is administered three times a day.

In some embodiments, compound 1, or a pharmaceutically acceptable salt thereof, is administered to a subject who is in a fasted state. A fasted state refers to a subject who has gone without food or fasted for a certain period of time. General fasting periods include at least 4 hours, at least 6 hours, at least 8 hours, at least 10 hours, at least 12 hours, at least 14 hours and at least 16 hours without food. In some embodiments, compound 1, or a pharmaceutically acceptable salt thereof, is administered to a subject who is in a fasted state for at least 8 hours. In other embodiments, compound 1, or a pharmaceutically acceptable salt thereof, is administered to a subject who is in a fasted state for at least 10 hours. In yet other embodiments, compound 1, or a pharmaceutically acceptable salt thereof, is administered to a subject who is in a fasted state for at least 12 hours. In other embodiments, compound 1, or a pharmaceutically acceptable salt thereof, is administered to a subject who has fasted overnight.

In other embodiments, compound 1, or a pharmaceutically acceptable salt thereof, is administered to a subject who is in a fed state. A fed state refers to a subject who has taken food or has had a meal. In certain embodiments, a composition is administered to a subject in a fed state 5 minutes post-meal, 10 minutes post-meal, 15 minutes post-meal, 20 minutes post-meal, 30 minutes post-meal, 40 minutes post-meal, 50 minutes post-meal, 1 hour post-meal, or 2 hours post-meal. In certain instances, compound 1, or a pharmaceutically acceptable salt thereof, is administered to a subject in a fed state 30 minutes post-meal. In other instances, compound 1, or a pharmaceutically acceptable salt thereof, is administered to a subject in a fed state 1 hour post-meal. In yet further embodiments, compound 1, or a pharmaceutically acceptable salt thereof, is administered to a subject with food.

The length of a treatment cycle depends on the treatment being given. In some embodiments, the length of a treatment cycle ranges from two to six weeks. In some embodiments, the length of a treatment cycle ranges from three to six weeks. In some embodiments, the length of a treatment cycle ranges from three to four weeks. In some embodiments, the length of a treatment cycle is three weeks (or 21 days). In some embodiments, the length of a treatment cycle is four weeks (28 days). In some embodiments, the length of a treatment cycle is five weeks (35 days). In some embodiments, the length of a treatment cycle is 56 days. In some embodiments, a treatment cycle lasts one, two, three, four, or five weeks. In some embodiments, a treatment cycle lasts three weeks. In some embodiments, a treatment cycle lasts four weeks. In some embodiments, a treatment cycle lasts five weeks. The number of treatment doses scheduled within each cycle also varies depending on the drugs being given.

In some embodiments of a method of treating cancer, compound 1, or a pharmaceutically acceptable salt thereof, is administered in 28-day cycles. In some embodiments of a method of treating cancer, compound 1, or a pharmaceutically acceptable salt thereof, is administered for multiple 28-day cycles. In some embodiments of a method of treating cancer, compound 1, or a pharmaceutically acceptable salt thereof, is administered for at least one 28-day cycle. In some embodiments of a method of treating cancer, compound 1, or a pharmaceutically acceptable salt thereof, is administered for at least two 28-day cycles. In some embodiments of a method of treating cancer, compound 1, or a pharmaceutically acceptable salt thereof, is administered for at least three 28-day cycles.

In some embodiments of a method of treating cancer, compound 1, or a pharmaceutically acceptable salt thereof, is administered on days 1-7 of each 28-day cycle. In some embodiments of a method of treating cancer, compound 1, or a pharmaceutically acceptable salt thereof, is administered on days 1-14 of each 28-day cycle. In some embodiments of a method of treating cancer, compound 1, or a pharmaceutically acceptable salt thereof, is administered on days 1-21 of each 28-day cycle. In some embodiments of a method of treating cancer, compound 1, or a pharmaceutically acceptable salt thereof, is administered on days 1-28 of each 28-day cycle.

In some embodiments of a method of treating cancer, compound 1, or a pharmaceutically acceptable salt thereof, is administered twice a day on day 1 of a 28-day cycle. In some embodiments of a method of treating cancer, compound 1, or a pharmaceutically acceptable salt thereof, is administered twice a day on day 8 of a 28-day cycle. In some embodiments of a method of treating cancer, compound 1, or a pharmaceutically acceptable salt thereof, is administered twice a day on day 15 of a 28-day cycle. In some embodiments of a method of treating cancer, compound 1, or a pharmaceutically acceptable salt thereof, is administered twice a day on day 22 of a 28-day cycle. In some embodiments of a method of treating cancer, compound 1, or a pharmaceutically acceptable salt thereof, is not administered twice a day on day 22 of a 28-day cycle.

In some embodiments of a method of treating cancer, compound 1, or a pharmaceutically acceptable salt thereof, is administered twice a day on day 1, day 8, and day 15 of a 28-day cycle.

In some embodiments of a method of treating cancer, compound 1, or a pharmaceutically acceptable salt thereof, is not administered on days 2-7, days 9-14, days 16-21, days 23-28 of a 28-day cycle.

In some embodiments of a method of treating cancer, compound 1, or a pharmaceutically acceptable salt thereof, is administered in 35-day cycles. In some embodiments of a method of treating cancer, compound 1, or a pharmaceutically acceptable salt thereof, is administered for multiple 35-day cycles. In some embodiments of a method of treating cancer, compound 1, or a pharmaceutically acceptable salt thereof, is administered for at least one 35-day cycle.

In some embodiments of a method of treating cancer, compound 1, or a pharmaceutically acceptable salt thereof, is administered on days 1-7 of each 35-day cycle. In some embodiments of a method of treating cancer, compound 1, or a pharmaceutically acceptable salt thereof, is administered on days 1-14 of each 35-day cycle. In some embodiments of a method of treating cancer, compound 1, or a pharmaceutically acceptable salt thereof, is administered on days 1-21 of each 35-day cycle. In some embodiments of a method of treating cancer, compound 1, or a pharmaceutically acceptable salt thereof, is administered on days 1-28 of each 35-day cycle. In some embodiments of a method of treating cancer, compound 1, or a pharmaceutically acceptable salt thereof, is administered on days 1-35 of each 35-day cycle.

In some embodiments of a method of treating cancer, compound 1, or a pharmaceutically acceptable salt thereof, is administered twice a day on day 1 of a 35-day cycle. In some embodiments of a method of treating cancer, compound 1, or a pharmaceutically acceptable salt thereof, is administered twice a day on day 8 of a 35-day cycle. In some embodiments of a method of treating cancer, compound 1, or a pharmaceutically acceptable salt thereof, is administered twice a day on day 15 of a 35-day cycle. In some embodiments of a method of treating cancer, compound 1, or a pharmaceutically acceptable salt thereof, is administered twice a day on day 22 of a 35-day cycle. In some embodiments of a method of treating cancer, compound 1, or a pharmaceutically acceptable salt thereof, is administered twice a day on day 29 of a 35-day cycle. In some embodiments of a method of treating cancer, compound 1, or a pharmaceutically acceptable salt thereof, is not administered twice a day on day 29 of a 35-day cycle.

In some embodiments of a method of treating cancer, compound 1, or a pharmaceutically acceptable salt thereof, is administered twice a day on day 1, day 8, day 15, and day 22 of a 35-day cycle.

In some embodiments of a method of treating cancer, compound 1, or a pharmaceutically acceptable salt thereof, is not administered on days 2-7, days 9-14, days 16-21, days 23-28, and days 30-35 of a 28-day cycle.

In some embodiments of a method of treating solid tumor, compound 1, or a pharmaceutically acceptable salt thereof, is administered in 28-day cycles. In some embodiments of a method of treating a solid tumor, compound 1, or a pharmaceutically acceptable salt thereof, is administered for multiple 28-day cycles. In some embodiments of a method of treating a solid tumor, compound 1, or a pharmaceutically acceptable salt thereof, is administered for at least one 28-day cycle. In some embodiments of a method of treating a solid tumor, compound 1, or a pharmaceutically acceptable salt thereof, is administered for at least two 28-day cycles. In some embodiments of a method of treating a solid tumor, compound 1, or a pharmaceutically acceptable salt thereof, is administered for at least three 28-day cycles.

In some embodiments of a method of treating a solid tumor, compound 1, or a pharmaceutically acceptable salt thereof, is administered on days 1-7 of each 28-day cycle. In some embodiments of a method of treating a solid tumor, compound 1, or a pharmaceutically acceptable salt thereof, is administered on days 1-14 of each 28-day cycle. In some embodiments of a method of treating a solid tumor, compound 1, or a pharmaceutically acceptable salt thereof, is administered on days 1-21 of each 28-day cycle. In some embodiments of a method of treating a solid tumor, compound 1, or a pharmaceutically acceptable salt thereof, is administered on days 1-28 of each 28-day cycle.

In some embodiments of a method of treating solid tumor, compound 1, or a pharmaceutically acceptable salt thereof, is administered twice a day on day 1 of a 28-day cycle. In some embodiments of a method of treating solid tumor, compound 1, or a pharmaceutically acceptable salt thereof, is administered twice a day on day 8 of a 28-day cycle. In some embodiments of a method of treating solid tumor, compound 1, or a pharmaceutically acceptable salt thereof, is administered twice a day on day 15 of a 28-day cycle. In some embodiments of a method of treating solid tumor, compound 1, or a pharmaceutically acceptable salt thereof, is administered twice a day on day 22 of a 28-day cycle. In some embodiments of a method of treating solid tumor, compound 1, or a pharmaceutically acceptable salt thereof, is not administered twice a day on day 22 of a 28-day cycle.

In some embodiments of a method of treating a solid tumor, compound 1, or a pharmaceutically acceptable salt thereof, is administered twice a day on day 1, day 8, and day 15 of a 28-day cycle.

In some embodiments of a method of treating a solid tumor, compound 1, or a pharmaceutically acceptable salt thereof, is not administered on days 2-7, days 9-14, days 16-21, days 23-28 of a 28-day cycle.

In some embodiments of a method of treating a solid tumor, compound 1, or a pharmaceutically acceptable salt thereof, is administered in 35-day cycles. In some embodiments of a method of treating a solid tumor, compound 1, or a pharmaceutically acceptable salt thereof, is administered for multiple 35-day cycles. In some embodiments of a method of treating a solid tumor, compound 1, or a pharmaceutically acceptable salt thereof, is administered for at least one 35-day cycle.

In some embodiments of a method of treating a solid tumor, compound 1, or a pharmaceutically acceptable salt thereof, is administered on days 1-7 of each 35-day cycle. In some embodiments of a method of treating a solid tumor, compound 1, or a pharmaceutically acceptable salt thereof, is administered on days 1-14 of each 35-day cycle. In some embodiments of a method of treating a solid tumor, compound 1, or a pharmaceutically acceptable salt thereof, is administered on days 1-21 of each 35-day cycle. In some embodiments of a method of treating a solid tumor, compound 1, or a pharmaceutically acceptable salt thereof, is administered on days 1-28 of each 35-day cycle. In some embodiments of a method of treating a solid tumor, compound 1, or a pharmaceutically acceptable salt thereof, is administered on days 1-35 of each 35-day cycle.

In some embodiments of a method of treating solid tumor, compound 1, or a pharmaceutically acceptable salt thereof, is administered twice a day on day 1 of a 35-day cycle. In some embodiments of a method of treating solid tumor, compound 1, or a pharmaceutically acceptable salt thereof, is administered twice a day on day 8 of a 35-day cycle. In some embodiments of a method of treating solid tumor, compound 1, or a pharmaceutically acceptable salt thereof, is administered twice a day on day 15 of a 35-day cycle. In some embodiments of a method of treating solid tumor, compound 1, or a pharmaceutically acceptable salt thereof, is administered twice a day on day 22 of a 35-day cycle. In some embodiments of a method of treating solid tumor, compound 1, or a pharmaceutically acceptable salt thereof, is administered twice a day on day 29 of a 35-day cycle. In some embodiments of a method of treating solid tumor, compound 1, or a pharmaceutically acceptable salt thereof, is not administered twice a day on day 29 of a 35-day cycle.

In some embodiments of a method of treating a solid tumor, compound 1, or a pharmaceutically acceptable salt thereof, is administered twice a day on day 1, day 8, day 15, and day 22 of a 35-day cycle.

In some embodiments of a method of treating a solid tumor, compound 1, or a pharmaceutically acceptable salt thereof, is not administered on days 2-7, days 9-14, days 16-21, days 23-28, and days 30-35 of a 28-day cycle.

Disclosed herein is a method of treating a solid tumor in a subject in need thereof, the method comprising administering to the subject in need thereof:
compound 1

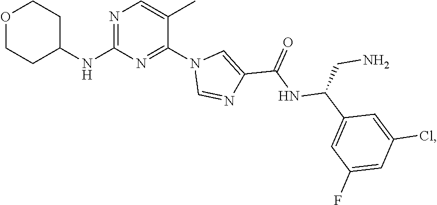 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
wherein compound 1 is administered twice a day, once a week (BID-QW).

In some embodiments of a method of treating a solid tumor, the salt of compound 1, is the mandelic acid salt.

In some embodiments of a method of treating a solid tumor, compound 1, or a pharmaceutically acceptable salt thereof, is administered for at least one 28-day cycle.

In some embodiments of a method of treating a solid tumor, compound 1, or a pharmaceutically acceptable salt thereof, is administered twice a day on day 1, day 8, day 15, and day 22 of a 28-day cycle.

In some embodiments of a method of treating a solid tumor, compound 1, or a pharmaceutically acceptable salt thereof, is administered twice a day on day 1, day 8, day 15 of a 28-day cycle.

In some embodiments of a method of treating a solid tumor, compound 1, or a pharmaceutically acceptable salt thereof, is not administered on days 2-7, days 9-14, days 16-21, and days 23-28 of a 28-day cycle.

In some embodiments of a method of treating a solid tumor, compound 1, or a pharmaceutically acceptable salt thereof, is administered for at least one 35-day cycle.

In some embodiments of a method of treating a solid tumor, compound 1, or a pharmaceutically acceptable salt thereof, is administered twice a day on day 1, day 8, day 15, and day 22 of a 35-day cycle.

In some embodiments of a method of treating a solid tumor, compound 1, or a pharmaceutically acceptable salt thereof, is not administered on days 2-7, days 9-14, days 16-21, days 23-28, and days 30-35 of a 28-day cycle.

In some embodiments of a method of treating a solid tumor, compound 1, or a pharmaceutically acceptable salt thereof, is administered in an amount that is between about 25 mg and about 300 mg twice a day, once a week (BID-QW).

In some embodiments of a method of treating a solid tumor, compound 1, or a pharmaceutically acceptable salt thereof, is administered in an amount that is between about 25 mg and about 250 mg twice a day, once a week (BID-QW).

In some embodiments of a method of treating a solid tumor, compound 1, or a pharmaceutically acceptable salt thereof, is administered in an amount that is between about 25 mg and about 150 mg twice a day, once a week (BID-QW).

In some embodiments of a method of treating a solid tumor, compound 1, or a pharmaceutically acceptable salt thereof, is administered in an amount that is about 25 mg, 50 mg, about 75 mg, about 100 mg, about 150 mg, about 175 mg, about 200 mg, about 225 mg, or about 250 mg twice a day, once a week (BID-QW).

In some embodiments of a method of treating a solid tumor, compound 1, or a pharmaceutically acceptable salt thereof, is administered in an amount that is about 25 mg, 50 mg, about 100 mg, or about 150 mg twice a day, once a week (BID-QW).

In some embodiments of a method of treating a solid tumor, the solid tumor comprises at least one cancer cell with one or more activating mutations in the MAPK pathway.

In some embodiments of a method of treating a solid tumor, the solid tumor is a mitogen-activated protein kinase (MAPK) pathway driven cancer.

In some embodiments of a method of treating a solid tumor, the solid tumor is a BRAF-driven cancer, HRAS-driven cancer, or a NRAS-driven cancer.

In some embodiments of a method of treating a solid tumor, the solid tumor comprises at least one cancer cell driven by deregulated ERK.

In some embodiments of a method of treating a solid tumor, the solid tumor has at least one mutation in RAS, RAF, or MEK.

In some embodiments of a method of treating a solid tumor, the subject has not previously received a MAPK pathway inhibitor (e.g., a KRAS inhibitor, a BRAF inhibitor, a MEK inhibitor, or an ERK inhibitor).

In some embodiments of a method of treating a solid tumor, the solid tumor is non-small cell lung cancer (NSCLC), melanoma, pancreatic cancer, or colorectal cancer (CRC).

In some embodiments of a method of treating a solid tumor, the solid tumor is a MAPKm/MAPKi-naïve pan tumor, MAPKm/MAPKi-naïve pancreatic cancer, MAPKm/MAPKi-naïve NSCLC, BRAFi-treated V600 NSCLC, BRAFi-treated V600 melanoma, or KRAS-treated G12C NSCLC.

In some embodiments of a method of treating a solid tumor, the subject in need thereof does not experience serious adverse events during the 28-day cycles.

In some embodiments of a method of treating a solid tumor, the subject in need thereof does not experience Grade 3, 4, or 5 adverse events during the 28-day cycles.

Disclosed herein is a method of treating non-small cell lung cancer (NSCLC) in a subject in need thereof; the method comprising administering to the subject in need thereof:
compound 1

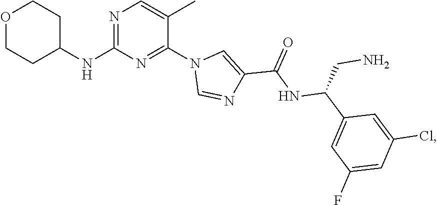 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
wherein the compound is administered twice a day, once a week (BID-QW).

In some embodiments of a method of treating non-small cell lung cancer (NSCLC), compound 1, or pharmaceutically acceptable salt thereof, is administered for at least one 28-day cycle.

In some embodiments of a method of treating non-small cell lung cancer (NSCLC), compound 1, or pharmaceutically acceptable salt thereof, is administered twice a day on day 1, day 8, day 15, and day 22 of a 28-day cycle.

In some embodiments of a method of treating non-small cell lung cancer (NSCLC), compound 1, or a pharmaceutically acceptable salt thereof, is not administered on days 2-7, days 9-14, and days 23-28 of a 28-day cycle.

In some embodiments of a method of treating non-small cell lung cancer (NSCLC), compound 1, or a pharmaceutically acceptable salt thereof, is administered for at least one 35-day cycle.

In some embodiments of a method of treating non-small cell lung cancer (NSCLC), compound 1, or a pharmaceutically acceptable salt thereof, is administered twice a day on day 1, day 8, day 15, and day 22 of a 35-day cycle.

In some embodiments of a method of treating non-small cell lung cancer (NSCLC), compound 1, or a pharmaceutically acceptable salt thereof, is not administered on days 2-7, days 9-14, days 16-21, days 23-28, and days 30-35 of a 28-day cycle.

In some embodiments of a method of treating non-small cell lung cancer (NSCLC), compound 1, or pharmaceutically acceptable salt thereof, is administered in an amount that is about 25 mg to 300 mg twice a day, once a week (BID-QW).

In some embodiments of a method of treating non-small cell lung cancer (NSCLC), compound 1, or pharmaceutically acceptable salt thereof, is administered in an amount that is about 25 mg to 250 mg twice a day, once a week (BID-QW).

In some embodiments of a method of treating non-small cell lung cancer (NSCLC), compound 1, or pharmaceutically acceptable salt thereof, is administered in an amount that is about 25 mg to 150 mg twice a day, once a week (BID-QW). In some embodiments of a method of treating non-small cell lung cancer (NSCLC), compound 1, or pharmaceutically acceptable salt thereof, is administered in an amount that is about 25 mg, about 50 mg, about 75 mg, about 100 mg, about 125 mg, about 150 mg, about 175 mg, about 200 mg, about 225 mg, or about 250 mg twice a day, once a week (BID-QW).

In some embodiments of a method of treating non-small cell lung cancer (NSCLC), compound 1, or a pharmaceutically acceptable salt thereof, is administered in an amount that is about 50 mg, about 100 mg, about 125 mg, or about 150 mg twice a day, once a week (BID-QW).

In some embodiments of a method of treating non-small cell lung cancer (NSCLC), the cancer is a mitogen-activated protein kinase (MAPK) pathway driven cancer.

In some embodiments of a method of treating non-small cell lung cancer (NSCLC), the cancer is a BRAF-driven cancer, HRAS-driven cancer, or a NRAS-driven cancer.

In some embodiments of a method of treating non-small cell lung cancer (NSCLC), the cancer comprises at least one cancer call driven by deregulated ERK.

In some embodiments of a method of treating non-small cell lung cancer (NSCLC), the cancer has at least one mutation in RAS, RAF, or MEK.

In some embodiments of a method of treating non-small cell lung cancer (NSCLC), the subject has not previously received a MAPK pathway inhibitors (e.g., a KRAS inhibitor, a BRAF inhibitor, a MEK inhibitor, or an ERK inhibitor).

In some embodiments of a method of treating non-small cell lung cancer (NSCLC), the cancer is a MAPKm/MAPKi-naïve pan tumor (−) CRC/NSCLC, MAPKm/MAPKi-naïve NSCLC, BRAFi-treated V600 NSCLC, or KRAS-treated G12C NSCLC.

In some embodiments of a method of treating non-small cell lung cancer (NSCLC), the subject in need thereof does not experience serious adverse events during the 28-day cycles.

In some embodiments of a method of treating non-small cell lung cancer (NSCLC), the subject in need thereof does not experience Grade 3, 4, or 5 adverse events during the 28-day cycles.

Disclosed herein is a method of treating melanoma in a subject in need thereof; the method comprising administering to the subject in need thereof:
compound 1

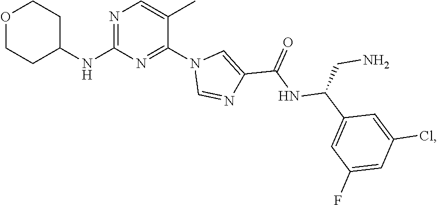 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
wherein the compound is administered twice a day, once a week (BID-QW).

In some embodiments of a method of treating melanoma, compound 1, or pharmaceutically acceptable salt thereof, is administered for at least one 28-day cycle.

In some embodiments of a method of treating melanoma, compound 1, or pharmaceutically acceptable salt thereof, is administered twice a day on day 1, day 8, day 15, and day 22 of a 28-day cycle.

In some embodiments of a method of treating melanoma, compound 1, or pharmaceutically acceptable salt thereof, is not administered on days 2-7, days 9-14, and days 23-28 of a 28-day cycle.

In some embodiments of a method of treating melanoma, compound 1, or a pharmaceutically acceptable salt thereof, is administered for at least one 35-day cycle.

In some embodiments of a method of treating melanoma, compound 1, or a pharmaceutically acceptable salt thereof, is administered twice a day on day 1, day 8, day 15, and day 22 of a 35-day cycle.

In some embodiments of a method of treating melanoma, compound 1, or a pharmaceutically acceptable salt thereof, is not administered on days 2-7, days 9-14, days 16-21, days 23-28, and days 30-35 of a 28-day cycle.

In some embodiments of a method of treating melanoma, compound 1, or pharmaceutically acceptable salt thereof, is administered in an amount that is about 25 mg to 300 mg twice a day, once a week (BID-QW).

In some embodiments of a method of treating melanoma, compound 1, or pharmaceutically acceptable salt thereof, is administered in an amount that is about 25 mg to 250 mg twice a day, once a week (BID-QW).

In some embodiments of a method of treating melanoma, compound 1, or pharmaceutically acceptable salt thereof, is administered in an amount that is about 25 mg to 150 mg twice a day, once a week (BID-QW).

In some embodiments of a method of treating melanoma, compound 1, or pharmaceutically acceptable salt thereof, is administered in an amount that is about 25 mg, about 50 mg, about 75 mg, about 100 mg, about 125 mg, about 150 mg, about 175 mg, about 200 mg, about 225 mg, or about 250 mg twice a day, once a week (BID-QW).

In some embodiments of a method of treating melanoma, compound 1, or a pharmaceutically acceptable salt thereof, is administered in an amount that is about 50 mg, about 100 mg, about 125 mg, or about 150 mg twice a day, once a week (BID-QW).

In some embodiments of a method of treating melanoma, the cancer is a mitogen-activated protein kinase (MAPK) pathway driven cancer.

In some embodiments of a method of treating melanoma, the cancer is a BRAF-driven cancer, HRAS-driven cancer, or a NRAS-driven cancer.

In some embodiments of a method of treating melanoma, the cancer comprises at least one cancer call driven by deregulated ERK.

In some embodiments of a method of treating melanoma, the cancer has at least one mutation in RAS, RAF, or MEK.

In some embodiments of a method of treating melanoma, the subject has not previously received a MAPK pathway inhibitor (e.g., a KRAS inhibitor, a BRAF inhibitor, a MEK inhibitor, or an ERK inhibitor).

In some embodiments of a method of treating melanoma, the cancer is a BRAFi-treated V600 melanoma.

In some embodiments of a method of treating melanoma, the subject in need thereof does not experience serious adverse events during the 28-day cycles.

In some embodiments of a method of treating melanoma, the subject in need thereof does not experience Grade 3, 4, or 5 adverse events during the 28-day cycles.

Disclosed herein is a method of treating pancreatic cancer in a subject in need thereof; the method comprising administering to the subject in need thereof:
compound 1

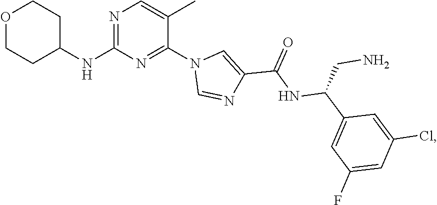 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
wherein the compound is administered twice a day, once a week (BID-QW).

In some embodiments of a method of treating pancreatic cancer, the pancreatic cancer is pancreatic ductal adenocarcinoma (PDAC).

In some embodiments of a method of treating pancreatic cancer, compound 1, or pharmaceutically acceptable salt thereof, is administered for at least one 28-day cycle.

In some embodiments of a method of treating pancreatic cancer, compound 1, or pharmaceutically acceptable salt thereof, is administered twice a day on day 1, day 8, day 15, and day 22 of a 28-day cycle.

In some embodiments of a method of treating pancreatic cancer, compound 1, or pharmaceutically acceptable salt thereof, is not administered on days 2-7, days 9-14, days 16-21, and days 23-28 of a 28-day cycle.

In some embodiments of a method of treating pancreatic cancer, compound 1, or a pharmaceutically acceptable salt thereof, is administered for at least one 35-day cycle.

In some embodiments of a method of treating pancreatic cancer, compound 1, or a pharmaceutically acceptable salt thereof, is administered twice a day on day 1, day 8, day 15, and day 22 of a 35-day cycle.

In some embodiments of a method of treating pancreatic cancer, compound 1, or a pharmaceutically acceptable salt thereof, is not administered on days 2-7, days 9-14, days 16-21, days 23-28, and days 30-35 of a 28-day cycle.

In some embodiments of a method of treating pancreatic cancer, compound 1, or pharmaceutically acceptable salt thereof, is administered in an amount that is about 25 mg to 300 mg twice a day, once a week (BID-QW).

In some embodiments of a method of treating pancreatic cancer, compound 1, or pharmaceutically acceptable salt thereof, is administered in an amount that is about 25 mg to 250 mg twice a day, once a week (BID-QW).

In some embodiments of a method of treating pancreatic cancer, compound 1, or pharmaceutically acceptable salt thereof, is administered in an amount that is about 25 mg to 150 mg twice a day, once a week (BID-QW).

In some embodiments of a method of treating pancreatic cancer, compound 1, or pharmaceutically acceptable salt thereof, is administered in an amount that is about 25 mg, about 50 mg, about 75 mg, about 100 mg, about 125 mg, about 150 mg, about 175 mg, about 200 mg, about 225 mg, or about 250 mg twice a day, once a week (BID-QW).

In some embodiments of a method of treating pancreatic cancer, compound 1, or a pharmaceutically acceptable salt thereof, is administered in an amount that is about 50 mg, about 100 mg, about 125 mg, or about 150 mg twice a day, once a week (BID-QW).

In some embodiments of a method of treating pancreatic cancer, the cancer is a mitogen-activated protein kinase (MAPK) pathway driven cancer.

In some embodiments of a method of treating pancreatic cancer, the cancer is a BRAF-driven cancer, HRAS-driven cancer, or a NRAS-driven cancer.

In some embodiments of a method of treating pancreatic cancer, the cancer comprises at least one cancer call driven by deregulated ERK.

In some embodiments of a method of treating pancreatic cancer, the cancer has at least one mutation in RAS, RAF, or MEK.

In some embodiments of a method of treating pancreatic cancer, the subject has not previously received a MAPK pathway inhibitor (e.g., a KRAS inhibitor, a BRAF inhibitor, a MEK inhibitor, or an ERK inhibitor).

In some embodiments of a method of treating pancreatic cancer, the cancer is a MAPKm/MAPKi-naïve pancreatic cancer.

In some embodiments of a method of treating pancreatic cancer, the subject in need thereof does not experience serious adverse events during the 28-day cycles.

In some embodiments of a method of treating pancreatic cancer, the subject in need thereof does not experience Grade 3, 4, or 5 adverse events during the 28-day cycles.

Disclosed herein is a method of treating colorectal cancer (CRC) in a subject in need thereof; the method comprising administering to the subject in need thereof:
compound 1

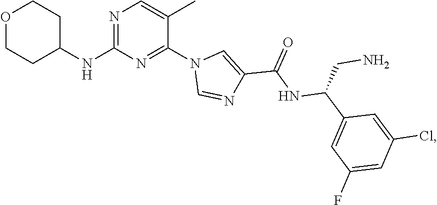 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
wherein the compound is administered twice a day, once a week (BID-QW).

In some embodiments of a method of treating colorectal cancer, compound 1, or pharmaceutically acceptable salt thereof, is administered for at least one 28-day cycle.

In some embodiments of a method of treating colorectal cancer, compound 1, or pharmaceutically acceptable salt thereof, is administered twice a day on day 1, day 8, day 15, and day 22 of a 28-day cycle.

In some embodiments of a method of treating colorectal cancer, compound 1, or pharmaceutically acceptable salt thereof, is not administered on days 2-7, days 9-14, days 16-21, and days 23-28 of a 28-day cycle.

In some embodiments of a method of treating colorectal cancer, compound 1, or a pharmaceutically acceptable salt thereof, is administered for at least one 35-day cycle.

In some embodiments of a method of treating colorectal cancer, compound 1, or a pharmaceutically acceptable salt thereof, is administered twice a day on day 1, day 8, day 15, and day 22 of a 35-day cycle.

In some embodiments of a method of treating colorectal cancer, compound 1, or a pharmaceutically acceptable salt thereof, is not administered on days 2-7, days 9-14, days 16-21, days 23-28, and days 30-35 of a 28-day cycle.

In some embodiments of a method of treating colorectal cancer, compound 1, or pharmaceutically acceptable salt thereof, is administered in an amount that is about 25 mg to 300 mg twice a day, once a week (BID-QW).

In some embodiments of a method of treating colorectal cancer, compound 1, or pharmaceutically acceptable salt thereof, is administered in an amount that is about 25 mg to 250 mg twice a day, once a week (BID-QW).

In some embodiments of a method of treating colorectal cancer, compound 1, or pharmaceutically acceptable salt thereof, is administered in an amount that is about 25 mg to 150 mg twice a day, once a week (BID-QW).

In some embodiments of a method of treating colorectal cancer, compound 1, or pharmaceutically acceptable salt thereof, is administered in an amount that is about 25 mg, about 50 mg, about 75 mg, about 100 mg, about 125 mg, about 150 mg, about 175 mg, about 200 mg, about 225 mg, or about 250 mg twice a day, once a week (BID-QW).

In some embodiments of a method of treating colorectal cancer, compound 1, or a pharmaceutically acceptable salt thereof, is administered in an amount that is about 50 mg, about 100 mg, about 125 mg, or about 150 mg twice a day, once a week (BID-QW).

In some embodiments of a method of treating colorectal cancer, the cancer is a mitogen-activated protein kinase (MAPK) pathway driven cancer.

In some embodiments of a method of treating colorectal cancer, the cancer is a BRAF-driven cancer, HRAS-driven cancer, or a NRAS-driven cancer.

In some embodiments of a method of treating colorectal cancer, the cancer comprises at least one cancer call driven by deregulated ERK.

In some embodiments of a method of treating colorectal cancer, the cancer has at least one mutation in RAS, RAF, or MEK.

In some embodiments of a method of treating colorectal cancer, the subject has not previously received a MAPK pathway inhibitor (e.g., a KRAS inhibitor, a BRAF inhibitor, a MEK inhibitor, or an ERK inhibitor).

In some embodiments of a method of treating colorectal cancer, the cancer is a MAPKm/MAPKi-naïve pan tumor.

In some embodiments of a method of treating colorectal cancer, the subject in need thereof does not experience serious adverse events during the 28-day cycles.

In some embodiments of a method of treating colorectal cancer, the subject in need thereof does not experience Grade 3, 4, or 5 adverse events during the 28-day cycles.

Disclosed herein is a method of treating cholangiocarcinoma cancer in a subject in need thereof; the method comprising administering to the subject in need thereof:
compound 1

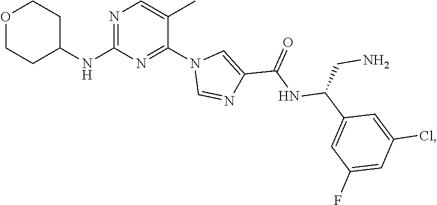 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
wherein the compound is administered twice a day, once a week (BID-QW).

In some embodiments of a method of treating cholangiocarcinoma cancer, compound 1, or pharmaceutically acceptable salt thereof, is administered for at least one 28-day cycle.

In some embodiments of a method of treating cholangiocarcinoma cancer, compound 1, or pharmaceutically acceptable salt thereof, is administered twice a day on day 1, day 8, day 15, and day 22 of a 28-day cycle.

In some embodiments of a method of treating cholangiocarcinoma cancer, compound 1, or pharmaceutically acceptable salt thereof, is not administered on days 2-7, days 9-14, days 16-21, and days 23-28 of a 28-day cycle.

In some embodiments of a method of treating cholangiocarcinoma cancer, compound 1, or a pharmaceutically acceptable salt thereof, is administered for at least one 35-day cycle.

In some embodiments of a method of treating cholangiocarcinoma cancer, compound 1, or a pharmaceutically acceptable salt thereof, is administered twice a day on day 1, day 8, day 15, and day 22 of a 35-day cycle.

In some embodiments of a method of treating cholangiocarcinoma cancer, compound 1, or a pharmaceutically acceptable salt thereof, is not administered on days 2-7, days 9-14, days 16-21, days 23-28, and days 30-35 of a 28-day cycle.

In some embodiments of a method of treating cholangiocarcinoma cancer, compound 1, or pharmaceutically acceptable salt thereof, is administered in an amount that is about 25 mg to 300 mg twice a day, once a week (BID-QW).

In some embodiments of a method of treating cholangiocarcinoma cancer, compound 1, or pharmaceutically acceptable salt thereof, is administered in an amount that is about 25 mg to 250 mg twice a day, once a week (BID-QW).

In some embodiments of a method of treating cholangiocarcinoma cancer, compound 1, or pharmaceutically acceptable salt thereof, is administered in an amount that is about 25 mg to 150 mg twice a day, once a week (BID-QW).

In some embodiments of a method of treating cholangiocarcinoma cancer, compound 1, or pharmaceutically acceptable salt thereof, is administered in an amount that is about 25 mg, about 50 mg, about 75 mg, about 100 mg, about 125 mg, about 150 mg, about 175 mg, about 200 mg, about 225 mg, or about 250 mg twice a day, once a week (BID-QW).

In some embodiments of a method of treating cholangiocarcinoma cancer, compound 1, or a pharmaceutically acceptable salt thereof, is administered in an amount that is about 50 mg, about 100 mg, about 125 mg, or about 150 mg twice a day, once a week (BID-QW).

In some embodiments of a method of treating cholangiocarcinoma cancer, the cancer is a mitogen-activated protein kinase (MAPK) pathway driven cancer.

In some embodiments of a method of treating cholangiocarcinoma cancer, the cancer is a BRAF-driven cancer, HRAS-driven cancer, or a NRAS-driven cancer.

In some embodiments of a method of treating cholangiocarcinoma cancer, the cancer comprises at least one cancer call driven by deregulated ERK.

In some embodiments of a method of treating cholangiocarcinoma cancer, the cancer has at least one mutation in RAS, RAF, or MEK.

In some embodiments of a method of treating cholangiocarcinoma cancer, the subject has not previously received a MAPK pathway inhibitor (e.g., a KRAS inhibitor, a BRAF inhibitor, a MEK inhibitor, or an ERK inhibitor).

In some embodiments of a method of treating cholangiocarcinoma cancer, the cancer is a MAPKm/MAPKi-naïve pan tumor.

In some embodiments of a method of treating cholangiocarcinoma cancer, the subject in need thereof does not experience serious adverse events during the 28-day cycles.

In some embodiments of a method of treating cholangiocarcinoma cancer, the subject in need thereof does not experience Grade 3, 4, or 5 adverse events during the 28-day cycles.

Disclosed herein is a method of treating appendiceal cancer in a subject in need thereof; the method comprising administering to the subject in need thereof:
compound 1

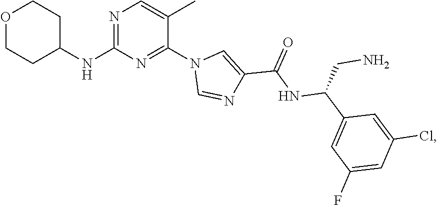 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
wherein the compound is administered twice a day, once a week (BID-QW).

In some embodiments of a method of treating appendiceal cancer, compound 1, or pharmaceutically acceptable salt thereof, is administered for at least one 28-day cycle.

In some embodiments of a method of treating appendiceal cancer, compound 1, or pharmaceutically acceptable salt thereof, is administered twice a day on day 1, day 8, day 15, and day 22 of a 28-day cycle.

In some embodiments of a method of treating appendiceal cancer, compound 1, or pharmaceutically acceptable salt thereof, is not administered on days 2-7, days 9-14, days 16-21, and days 23-28 of a 28-day cycle.

In some embodiments of a method of treating appendiceal cancer, compound 1, or a pharmaceutically acceptable salt thereof, is administered for at least one 35-day cycle.

In some embodiments of a method of treating appendiceal cancer, compound 1, or a pharmaceutically acceptable salt thereof, is administered twice a day on day 1, day 8, day 15, and day 22 of a 35-day cycle.

In some embodiments of a method of treating appendiceal cancer, compound 1, or a pharmaceutically acceptable salt thereof, is not administered on days 2-7, days 9-14, days 16-21, days 23-28, and days 30-35 of a 28-day cycle.

In some embodiments of a method of treating appendiceal cancer, compound 1, or pharmaceutically acceptable salt thereof, is administered in an amount that is about 25 mg to 300 mg twice a day, once a week (BID-QW).

In some embodiments of a method of treating appendiceal cancer, compound 1, or pharmaceutically acceptable salt thereof, is administered in an amount that is about 25 mg to 250 mg twice a day, once a week (BID-QW).

In some embodiments of a method of treating appendiceal cancer, compound 1, or pharmaceutically acceptable salt thereof, is administered in an amount that is about 25 mg to 150 mg twice a day, once a week (BID-QW).

In some embodiments of a method of treating appendiceal cancer, compound 1, or pharmaceutically acceptable salt thereof, is administered in an amount that is about 25 mg, about 50 mg, about 75 mg, about 100 mg, about 125 mg, about 150 mg, about 175 mg, about 200 mg, about 225 mg, or about 250 mg twice a day, once a week (BID-QW).

In some embodiments of a method of treating appendiceal cancer, compound 1, or a pharmaceutically acceptable salt thereof, is administered in an amount that is about 50 mg, about 100 mg, about 125 mg, or about 150 mg twice a day, once a week (BID-QW).

In some embodiments of a method of treating appendiceal cancer, the cancer is a mitogen-activated protein kinase (MAPK) pathway driven cancer.

In some embodiments of a method of treating appendiceal cancer, the cancer is a BRAF-driven cancer, HRAS-driven cancer, or a NRAS-driven cancer.

In some embodiments of a method of treating appendiceal cancer, the cancer comprises at least one cancer call driven by deregulated ERK.

In some embodiments of a method of treating appendiceal cancer, the cancer has at least one mutation in RAS, RAF, or MEK.

In some embodiments of a method of treating appendiceal cancer, the subject has not previously received a MAPK pathway inhibitor (e.g., a KRAS inhibitor, a BRAF inhibitor, a MEK inhibitor, or an ERK inhibitor).

In some embodiments of a method of treating appendiceal cancer, the cancer is a MAPKm/MAPKi-naïve pan tumor.

In some embodiments of a method of treating appendiceal cancer, the subject in need thereof does not experience serious adverse events during the 28-day cycles.

In some embodiments of a method of treating appendiceal cancer, the subject in need thereof does not experience Grade 3, 4, or 5 adverse events during the 28-day cycles.

Disclosed herein is a method of treating gastric cancer in a subject in need thereof; the method comprising administering to the subject in need thereof:
compound 1

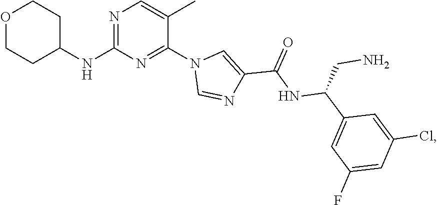 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
wherein the compound is administered twice a day, once a week (BID-QW).

In some embodiments of a method of treating gastric cancer, compound 1, or pharmaceutically acceptable salt thereof, is administered for at least one 28-day cycle.

In some embodiments of a method of treating gastric cancer, compound 1, or pharmaceutically acceptable salt thereof, is administered twice a day on day 1, day 8, day 15, and day 22 of a 28-day cycle.

In some embodiments of a method of treating gastric cancer, compound 1, or pharmaceutically acceptable salt thereof, is not administered on days 2-7, days 9-14, days 16-21, and days 23-28 of a 28-day cycle.

In some embodiments of a method of treating gastric cancer, compound 1, or a pharmaceutically acceptable salt thereof, is administered for at least one 35-day cycle.

In some embodiments of a method of treating gastric cancer, compound 1, or a pharmaceutically acceptable salt thereof, is administered twice a day on day 1, day 8, day 15, and day 22 of a 35-day cycle.

In some embodiments of a method of treating gastric cancer, compound 1, or a pharmaceutically acceptable salt thereof, is not administered on days 2-7, days 9-14, days 16-21, days 23-28, and days 30-35 of a 28-day cycle.

In some embodiments of a method of treating gastric cancer, compound 1, or pharmaceutically acceptable salt thereof, is administered in an amount that is about 25 mg to 300 mg twice a day, once a week (BID-QW).

In some embodiments of a method of treating gastric cancer, compound 1, or pharmaceutically acceptable salt thereof, is administered in an amount that is about 25 mg to 250 mg twice a day, once a week (BID-QW).

In some embodiments of a method of treating gastric cancer, compound 1, or pharmaceutically acceptable salt thereof, is administered in an amount that is about 25 mg to 150 mg twice a day, once a week (BID-QW).

In some embodiments of a method of treating gastric cancer, compound 1, or pharmaceutically acceptable salt thereof, is administered in an amount that is about 25 mg, about 50 mg, about 75 mg, about 100 mg, about 125 mg, about 150 mg, about 175 mg, about 200 mg, about 225 mg, or about 250 mg twice a day, once a week (BID-QW).

In some embodiments of a method of treating gastric cancer, compound 1, or a pharmaceutically acceptable salt thereof, is administered in an amount that is about 50 mg, about 100 mg, about 125 mg, or about 150 mg twice a day, once a week (BID-QW).

In some embodiments of a method of treating gastric cancer, the cancer is a mitogen-activated protein kinase (MAPK) pathway driven cancer.

In some embodiments of a method of treating gastric cancer, the cancer is a BRAF-driven cancer, HRAS-driven cancer, or a NRAS-driven cancer.

In some embodiments of a method of treating gastric cancer, the cancer comprises at least one cancer call driven by deregulated ERK.

In some embodiments of a method of treating gastric cancer, the cancer has at least one mutation in RAS, RAF, or MEK.

In some embodiments of a method of treating gastric cancer, the subject has not previously received a MAPK pathway inhibitor (e.g., a KRAS inhibitor, a BRAF inhibitor, a MEK inhibitor, or an ERK inhibitor).

In some embodiments of a method of treating gastric cancer, the cancer is a MAPKm/MAPKi-naïve pan tumor.

In some embodiments of a method of treating gastric cancer, the subject in need thereof does not experience serious adverse events during the 28-day cycles.

In some embodiments of a method of treating gastric cancer, the subject in need thereof does not experience Grade 3, 4, or 5 adverse events during the 28-day cycles.

Disclosed herein is a method of treating esophageal cancer in a subject in need thereof; the method comprising administering to the subject in need thereof:
compound 1

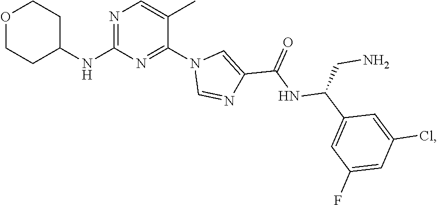 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
wherein the compound is administered twice a day, once a week (BID-QW).

In some embodiments of a method of treating esophageal cancer, compound 1, or pharmaceutically acceptable salt thereof, is administered for at least one 28-day cycle.

In some embodiments of a method of treating esophageal cancer, compound 1, or pharmaceutically acceptable salt thereof, is administered twice a day on day 1, day 8, day 15, and day 22 of a 28-day cycle.

In some embodiments of a method of treating esophageal cancer, compound 1, or pharmaceutically acceptable salt thereof, is not administered on days 2-7, days 9-14, days 16-21, and days 23-28 of a 28-day cycle.

In some embodiments of a method of treating esophageal cancer, compound 1, or a pharmaceutically acceptable salt thereof, is administered for at least one 35-day cycle.

In some embodiments of a method of treating esophageal cancer, compound 1, or a pharmaceutically acceptable salt thereof, is administered twice a day on day 1, day 8, day 15, and day 22 of a 35-day cycle.

In some embodiments of a method of treating esophageal cancer, compound 1, or a pharmaceutically acceptable salt thereof, is not administered on days 2-7, days 9-14, days 16-21, days 23-28, and days 30-35 of a 28-day cycle.

In some embodiments of a method of treating esophageal cancer, compound 1, or pharmaceutically acceptable salt thereof, is administered in an amount that is about 25 mg to 300 mg twice a day, once a week (BID-QW).

In some embodiments of a method of treating esophageal cancer, compound 1, or pharmaceutically acceptable salt thereof, is administered in an amount that is about 25 mg to 250 mg twice a day, once a week (BID-QW).

In some embodiments of a method of treating esophageal cancer, compound 1, or pharmaceutically acceptable salt thereof, is administered in an amount that is about 25 mg to 150 mg twice a day, once a week (BID-QW).

In some embodiments of a method of treating esophageal cancer, compound 1, or pharmaceutically acceptable salt thereof, is administered in an amount that is about 25 mg, about 50 mg, about 75 mg, about 100 mg, about 125 mg, about 150 mg, about 175 mg, about 200 mg, about 225 mg, or about 250 mg twice a day, once a week (BID-QW).

In some embodiments of a method of treating esophageal cancer, compound 1, or a pharmaceutically acceptable salt thereof, is administered in an amount that is about 50 mg, about 100 mg, about 125 mg, or about 150 mg twice a day, once a week (BID-QW).

In some embodiments of a method of treating esophageal cancer, the cancer is a mitogen-activated protein kinase (MAPK) pathway driven cancer.

In some embodiments of a method of treating esophageal cancer, the cancer is a BRAF-driven cancer, HRAS-driven cancer, or a NRAS-driven cancer.

In some embodiments of a method of treating esophageal cancer, the cancer comprises at least one cancer call driven by deregulated ERK.

In some embodiments of a method of treating esophageal cancer, the cancer has at least one mutation in RAS, RAF, or MEK.

In some embodiments of a method of treating esophageal cancer, the subject has not previously received a MAPK pathway inhibitor (e.g., a KRAS inhibitor, a BRAF inhibitor, a MEK inhibitor, or an ERK inhibitor).

In some embodiments of a method of treating esophageal cancer, the cancer is a MAPKm/MAPKi-naïve pan tumor.

In some embodiments of a method of treating esophageal cancer, the subject in need thereof does not experience serious adverse events during the 28-day cycles.

In some embodiments of a method of treating esophageal cancer, the subject in need thereof does not experience Grade 3, 4, or 5 adverse events during the 28-day cycles.

Disclosed herein is a method of treating head and neck cancer in a subject in need thereof; the method comprising administering to the subject in need thereof:
compound 1

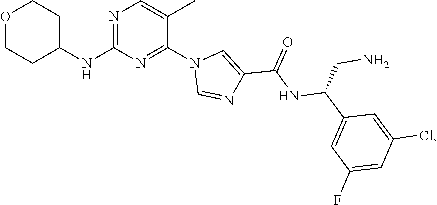 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
wherein the compound is administered twice a day, once a week (BID-QW).

In some embodiments of a method of treating head and neck cancer, compound 1, or pharmaceutically acceptable salt thereof, is administered for at least one 28-day cycle.

In some embodiments of a method of treating head and neck cancer, compound 1, or pharmaceutically acceptable salt thereof, is administered twice a day on day 1, day 8, day 15, and day 22 of a 28-day cycle.

In some embodiments of a method of treating head and neck cancer, compound 1, or pharmaceutically acceptable salt thereof, is not administered on days 2-7, days 9-14, days 16-21, and days 23-28 of a 28-day cycle.

In some embodiments of a method of treating head and neck cancer, compound 1, or a pharmaceutically acceptable salt thereof, is administered for at least one 35-day cycle.

In some embodiments of a method of treating head and neck cancer, compound 1, or a pharmaceutically acceptable salt thereof, is administered twice a day on day 1, day 8, day 15, and day 22 of a 35-day cycle.

In some embodiments of a method of treating head and neck cancer, compound 1, or a pharmaceutically acceptable salt thereof, is not administered on days 2-7, days 9-14, days 16-21, days 23-28, and days 30-35 of a 28-day cycle.

In some embodiments of a method of treating head and neck cancer, compound 1, or pharmaceutically acceptable salt thereof, is administered in an amount that is about 25 mg to 300 mg twice a day, once a week (BID-QW).

In some embodiments of a method of treating head and neck cancer, compound 1, or pharmaceutically acceptable salt thereof, is administered in an amount that is about 25 mg to 250 mg twice a day, once a week (BID-QW).

In some embodiments of a method of treating head and neck cancer, compound 1, or pharmaceutically acceptable salt thereof, is administered in an amount that is about 25 mg to 150 mg twice a day, once a week (BID-QW).

In some embodiments of a method of treating head and neck cancer, compound 1, or pharmaceutically acceptable salt thereof, is administered in an amount that is about 25 mg, about 50 mg, about 75 mg, about 100 mg, about 125 mg, about 150 mg, about 175 mg, about 200 mg, about 225 mg, or about 250 mg twice a day, once a week (BID-QW).

In some embodiments of a method of treating head and neck cancer, compound 1, or a pharmaceutically acceptable salt thereof, is administered in an amount that is about 50 mg, about 100 mg, about 125 mg, or about 150 mg twice a day, once a week (BID-QW).

In some embodiments of a method of treating head and neck cancer, the cancer is a mitogen-activated protein kinase (MAPK) pathway driven cancer.

In some embodiments of a method of treating head and neck cancer, the cancer is a BRAF-driven cancer, HRAS-driven cancer, or a NRAS-driven cancer.

In some embodiments of a method of treating head and neck cancer, the cancer comprises at least one cancer call driven by deregulated ERK.

In some embodiments of a method of treating head and neck cancer, the cancer has at least one mutation in RAS, RAF, or MEK.

In some embodiments of a method of treating head and neck cancer, the subject has not previously received a MAPK pathway inhibitor (e.g., a KRAS inhibitor, a BRAF inhibitor, a MEK inhibitor, or an ERK inhibitor).

In some embodiments of a method of treating head and neck cancer, the cancer is a MAPKm/MAPKi-naïve pan tumor.

In some embodiments of a method of treating head and neck cancer, the subject in need thereof does not experience serious adverse events during the 28-day cycles.

In some embodiments of a method of treating head and neck cancer, the subject in need thereof does not experience Grade 3, 4, or 5 adverse events during the 28-day cycles.

Disclosed herein is a method of treating ovarian cancer in a subject in need thereof; the method comprising administering to the subject in need thereof:
compound 1

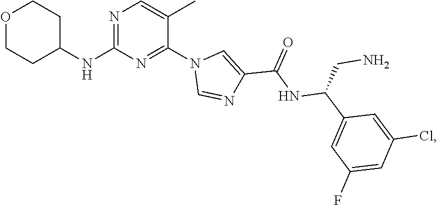 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
wherein the compound is administered twice a day, once a week (BID-QW).

In some embodiments of a method of treating ovarian cancer, compound 1, or pharmaceutically acceptable salt thereof, is administered for at least one 28-day cycle.

In some embodiments of a method of treating ovarian cancer, compound 1, or pharmaceutically acceptable salt thereof, is administered twice a day on day 1, day 8, day 15, and day 22 of a 28-day cycle.

In some embodiments of a method of treating ovarian cancer, compound 1, or pharmaceutically acceptable salt thereof, is not administered on days 2-7, days 9-14, days 16-21, and days 23-28 of a 28-day cycle.

In some embodiments of a method of treating ovarian cancer, compound 1, or a pharmaceutically acceptable salt thereof, is administered for at least one 35-day cycle.

In some embodiments of a method of treating ovarian cancer, compound 1, or a pharmaceutically acceptable salt thereof, is administered twice a day on day 1, day 8, day 15, and day 22 of a 35-day cycle.

In some embodiments of a method of treating ovarian cancer, compound 1, or a pharmaceutically acceptable salt thereof, is not administered on days 2-7, days 9-14, days 16-21, days 23-28, and days 30-35 of a 28-day cycle.

In some embodiments of a method of treating ovarian cancer, compound 1, or pharmaceutically acceptable salt thereof, is administered in an amount that is about 25 mg to 300 mg twice a day, once a week (BID-QW).

In some embodiments of a method of treating ovarian cancer, compound 1, or pharmaceutically acceptable salt thereof, is administered in an amount that is about 25 mg to 250 mg twice a day, once a week (BID-QW).

In some embodiments of a method of treating ovarian cancer, compound 1, or pharmaceutically acceptable salt thereof, is administered in an amount that is about 25 mg to 150 mg twice a day, once a week (BID-QW).

In some embodiments of a method of treating ovarian cancer, compound 1, or pharmaceutically acceptable salt thereof, is administered in an amount that is about 25 mg, about 50 mg, about 75 mg, about 100 mg, about 125 mg, about 150 mg, about 175 mg, about 200 mg, about 225 mg, or about 250 mg twice a day, once a week (BID-QW).

In some embodiments of a method of treating ovarian cancer, compound 1, or a pharmaceutically acceptable salt thereof, is administered in an amount that is about 50 mg, about 100 mg, about 125 mg, or about 150 mg twice a day, once a week (BID-QW).

In some embodiments of a method of treating ovarian cancer, the cancer is a mitogen-activated protein kinase (MAPK) pathway driven cancer.

In some embodiments of a method of treating ovarian cancer, the cancer is a BRAF-driven cancer, HRAS-driven cancer, or a NRAS-driven cancer.

In some embodiments of a method of treating ovarian cancer, the cancer comprises at least one cancer call driven by deregulated ERK.

In some embodiments of a method of treating ovarian cancer, the cancer has at least one mutation in RAS, RAF, or MEK.

In some embodiments of a method of treating ovarian cancer, the subject has not previously received a MAPK pathway inhibitor (e.g., a KRAS inhibitor, a BRAF inhibitor, a MEK inhibitor, or an ERK inhibitor).

In some embodiments of a method of treating ovarian cancer, the cancer is a MAPKm/MAPKi-naïve pan tumor.

In some embodiments of a method of treating ovarian cancer, the subject in need thereof does not experience serious adverse events during the 28-day cycles.

In some embodiments of a method of treating ovarian cancer, the subject in need thereof does not experience Grade 3, 4, or 5 adverse events during the 28-day cycles.

Disclosed herein is a method of treating uterine cancer in a subject in need thereof; the method comprising administering to the subject in need thereof:
compound 1

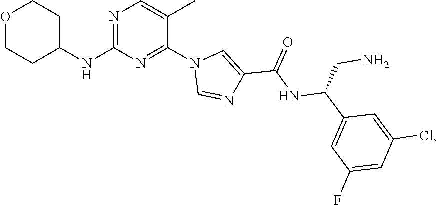 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
wherein the compound is administered twice a day, once a week (BID-QW).

In some embodiments of a method of treating uterine cancer, compound 1, or pharmaceutically acceptable salt thereof, is administered for at least one 28-day cycle.

In some embodiments of a method of treating uterine cancer, compound 1, or pharmaceutically acceptable salt thereof, is administered twice a day on day 1, day 8, day 15, and day 22 of a 28-day cycle.

In some embodiments of a method of treating uterine cancer, compound 1, or pharmaceutically acceptable salt thereof, is not administered on days 2-7, days 9-14, days 16-21, and days 23-28 of a 28-day cycle.

In some embodiments of a method of treating uterine cancer, compound 1, or a pharmaceutically acceptable salt thereof, is administered for at least one 35-day cycle.

In some embodiments of a method of treating uterine cancer, compound 1, or a pharmaceutically acceptable salt thereof, is administered twice a day on day 1, day 8, day 15, and day 22 of a 35-day cycle.

In some embodiments of a method of treating uterine cancer, compound 1, or a pharmaceutically acceptable salt thereof, is not administered on days 2-7, days 9-14, days 16-21, days 23-28, and days 30-35 of a 28-day cycle.

In some embodiments of a method of treating uterine cancer, compound 1, or pharmaceutically acceptable salt thereof, is administered in an amount that is about 25 mg to 300 mg twice a day, once a week (BID-QW).

In some embodiments of a method of treating uterine cancer, compound 1, or pharmaceutically acceptable salt thereof, is administered in an amount that is about 25 mg to 250 mg twice a day, once a week (BID-QW).

In some embodiments of a method of treating uterine cancer, compound 1, or pharmaceutically acceptable salt thereof, is administered in an amount that is about 25 mg to 150 mg twice a day, once a week (BID-QW).

In some embodiments of a method of treating uterine cancer, compound 1, or pharmaceutically acceptable salt thereof, is administered in an amount that is about 25 mg, about 50 mg, about 75 mg, about 100 mg, about 125 mg, about 150 mg, about 175 mg, about 200 mg, about 225 mg, or about 250 mg twice a day, once a week (BID-QW).

In some embodiments of a method of treating uterine cancer, compound 1, or a pharmaceutically acceptable salt thereof, is administered in an amount that is about 50 mg, about 100 mg, about 125 mg, or about 150 mg twice a day, once a week (BID-QW).

In some embodiments of a method of treating uterine cancer, the cancer is a mitogen-activated protein kinase (MAPK) pathway driven cancer.

In some embodiments of a method of treating uterine cancer, the cancer is a BRAF-driven cancer, HRAS-driven cancer, or a NRAS-driven cancer.

In some embodiments of a method of treating uterine cancer, the cancer comprises at least one cancer call driven by deregulated ERK.

In some embodiments of a method of treating uterine cancer, the cancer has at least one mutation in RAS, RAF, or MEK.

In some embodiments of a method of treating uterine cancer, the subject has not previously received a MAPK pathway inhibitor (e.g., a KRAS inhibitor, a BRAF inhibitor, a MEK inhibitor, or an ERK inhibitor).

In some embodiments of a method of treating uterine cancer, the cancer is a MAPKm/MAPKi-naïve pan tumor.

In some embodiments of a method of treating uterine cancer, the subject in need thereof does not experience serious adverse events during the 28-day cycles.

In some embodiments of a method of treating uterine cancer, the subject in need thereof does not experience Grade 3, 4, or 5 adverse events during the 28-day cycles.

Disclosed herein is a method of treating acute myeloid leukemia (AML) in a subject in need thereof; the method comprising administering to the subject in need thereof:
compound 1

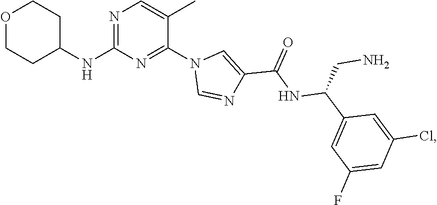 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
wherein the compound is administered twice a day, once a week (BID-QW).

In some embodiments of a method of treating acute myeloid leukemia, compound 1, or pharmaceutically acceptable salt thereof, is administered for at least one 28-day cycle.

In some embodiments of a method of treating acute myeloid leukemia, compound 1, or pharmaceutically acceptable salt thereof, is administered twice a day on day 1, day 8, day 15, and day 22 of a 28-day cycle.

In some embodiments of a method of treating acute myeloid leukemia, compound 1, or pharmaceutically acceptable salt thereof, is not administered on days 2-7, days 9-14, days 16-21, and days 23-28 of a 28-day cycle.

In some embodiments of a method of treating acute myeloid leukemia, compound 1, or a pharmaceutically acceptable salt thereof, is administered for at least one 35-day cycle.

In some embodiments of a method of treating acute myeloid leukemia, compound 1, or a pharmaceutically acceptable salt thereof, is administered twice a day on day 1, day 8, day 15, and day 22 of a 35-day cycle.

In some embodiments of a method of treating acute myeloid leukemia, compound 1, or a pharmaceutically acceptable salt thereof, is not administered on days 2-7, days 9-14, days 16-21, days 23-28, and days 30-35 of a 28-day cycle.

In some embodiments of a method of treating acute myeloid leukemia, compound 1, or pharmaceutically acceptable salt thereof, is administered in an amount that is about 25 mg to 300 mg twice a day, once a week (BID-QW).

In some embodiments of a method of treating acute myeloid leukemia, compound 1, or pharmaceutically acceptable salt thereof, is administered in an amount that is about 25 mg to 250 mg twice a day, once a week (BID-QW).

In some embodiments of a method of treating acute myeloid leukemia, compound 1, or pharmaceutically acceptable salt thereof, is administered in an amount that is about 25 mg to 150 mg twice a day, once a week (BID-QW).

In some embodiments of a method of treating acute myeloid leukemia, compound 1, or pharmaceutically acceptable salt thereof, is administered in an amount that is about 25 mg, about 50 mg, about 75 mg, about 100 mg, about 125 mg, about 150 mg, about 175 mg, about 200 mg, about 225 mg, or about 250 mg twice a day, once a week (BID-QW).

In some embodiments of a method of treating acute myeloid leukemia, compound 1, or a pharmaceutically acceptable salt thereof, is administered in an amount that is about 50 mg, about 100 mg, about 125 mg, or about 150 mg twice a day, once a week (BID-QW).

In some embodiments of a method of treating acute myeloid leukemia, the cancer is a mitogen-activated protein kinase (MAPK) pathway driven cancer.

In some embodiments of a method of treating acute myeloid leukemia, the cancer is a BRAF-driven cancer, HRAS-driven cancer, or a NRAS-driven cancer.

In some embodiments of a method of treating acute myeloid leukemia, the cancer comprises at least one cancer call driven by deregulated ERK.

In some embodiments of a method of treating acute myeloid leukemia, the cancer has at least one mutation in RAS, RAF, or MEK.

In some embodiments of a method of treating acute myeloid leukemia, the subject has not previously received a MAPK pathway inhibitor (e.g., a KRAS inhibitor, a BRAF inhibitor, a MEK inhibitor, or an ERK inhibitor).

In some embodiments of a method of treating acute myeloid leukemia, the cancer is a MAPKm/MAPKi-naïve pan tumor.

In some embodiments of a method of treating acute myeloid leukemia, the subject in need thereof does not experience serious adverse events during the 28-day cycles.

In some embodiments of a method of treating acute myeloid leukemia, the subject in need thereof does not experience Grade 3, 4, or 5 adverse events during the 28-day cycles.

### EXAMPLES

### Example 1—Open-Label Phase 1b/2 Study of Compound 1 in Patients with Advanced or Metastatic Solid Tumors

### Study Design

Compound 1 in the form of a pharmaceutical composition was administered as monotherapy to subjects having solid tumors in an open-label, multi-center clinical study. After the screening period, eligible subjects were enrolled and treated with the pharmaceutical composition comprising compound 1 as monotherapy until disease progression, unacceptable toxicity, or meeting another criterion for stopping treatment.

The study commenced with Part A dose escalation of Compound 1 administered BID-QW as monotherapy, and Part B, further characterization of the activity of Compound 1 administered at the QW RD of 250 mg, to be opened simultaneously. Once the monotherapy MTD or RD was determined from Part A, then dose expansion of Compound 1 monotherapy BID-QW commenced in Part C pending an acceptable safety and PK/PD profile.

All patients underwent screening assessments within 30 days prior to the first dose of compound 1. Each treatment cycle consists of a 4-week (28-day) treatment period.

This study was divided into 3 parts, as outlined below.

### Part A

Part A evaluated Compound 1 as monotherapy administered BID-QW over 28-day treatment cycles using a rolling six dose escalation design to enroll patients into the study.

Dose levels tested were 50, 100, 125 mg BID dosed on Day 1, QW. The starting dose was 50 mg BID-QW, which is equivalent to approximately half the weekly RD of 250 mg QW. Treatment cycles were every 4 weeks (28 days).

The rolling six design was used for enrollment into Part A. Two to six patients can be concurrently enrolled into a dose level, depending upon the number of patients enrolled at the current dose level, the number of patients who have experienced a dose limiting toxicity (DLT) at the current dose level, and the number of patients pending full DLT evaluation at the current dose level. Accrual was suspended when a cohort of 6 has enrolled or when the study endpoints was met. Dose level assignment was based on the number of patients currently enrolled in the cohort, the number of DLTs observed, and the number of patients at risk for developing a DLT (i.e., patients enrolled but who have not completed the DLT evaluation period). For example, if 3 patients were enrolled into the same dose cohort and each has cleared the DLT evaluation period with no reported DLTs, then the dose was escalated, and a subsequent patient could be enrolled at the next higher dose level. If any of the first 3 patients did not cleared the DLT evaluation period, or if one DLT has been observed, then a subsequent patient was entered at the same dose level. Lastly, if 2 or more DLTs were observed, the dose level was deescalated. This process was repeated for patients 5 and 6.

In place of suspending accrual after every 3 patients, accrual was only suspended when a cohort of 6 was filled. When patients were unevaluable for DLT, they were replaced with the next available patient if escalation or de-escalation rules was not fulfilled at the time the next available patient was enrolled onto the study. The rolling six design was subject to an interim safety review to ensure that no more subjects than necessary are potentially being exposed to an unsafe dose.

Sequential dose levels received escalating doses of Compound 1 until at least one of the following criteria are met:
a. The MTD was exceeded
b. The RD was defined
c. PK futility (defined as no substantive increase in exposure despite administration of increasing doses)
d. The Sponsor elected to halt further dose escalation (e.g., based on robust antitumor activity at the current dose levels)

Prior to meeting at least one of the above criteria, dose escalation proceeded independently within Part A.

### Part B (Dose Expansion, 250 Mg QW Schedule)

Part B evaluates safety, PK, PD, and clinical activity of compound 1.

A pilot food effect sub-study was conducted in approximately 10 patients at designated sites to evaluate the effect of high-fat meal on the PK of compound 1.

### Part C (Dose Expansion, BID-QW Schedule)

Part C evaluates safety, PK, PD, and preliminary clinical activity of Compound 1 in the following groups:
Group 1: Patients with solid tumors with any documented activating mutation(s) in the MAPK pathway, excluding CRC and NSCLC, who have not previously received a MAPK inhibitor (Stage I up to 16, Stage II up to 15)
Group 2: Patients with pancreatic cancer with any documented activating mutation(s) in the MAPK pathway who have not previously received a MAPK inhibitor (Stage I up to 16, Stage II up to 15)
Group 3: Patients with NSCLC with any documented activating mutation(s) in the MAPK pathway who have not previously received a MAPK inhibitor (Stage I up to 16, Stage II up to 15)
Group 4: Patients with documented BRAF V600 melanomas who have previously received a BRAF inhibitor (Stage I up to 16, Stage II up to 15)
Group 5: Patients with documented BRAF V600 NSCLC who have previously received a BRAF inhibitor (Stage I up to 160, Stage II up to 15)
Group 6: Patients with KRAS G12C NSCLC who have previously received a G12C inhibitor (Stage I up to 16, Stage II up to 15)

For each group defined above that pass the Stage 1 gate of an ORR ≥10% then the Stage II patients were enrolled.

### Study Treatment

In Part A, Compound 1 was administered as a monotherapy BD-QD over 28-day cycles. Part A consists of several dose escalation cohorts. The starting dose for Part A was 50 mg BID-QW.

In Part B, Compound 1 was administered as a monotherapy QW over continuous 4-week cycles at a dose of 250 mg QW.

In Part C, Compound 1 was administered as a monotherapy BID-QW over 28-day cycles. The dose level administered in Part C was the monotherapy RD as determined in Part A.

Within these parts, the sample sizes have been calculated based upon enrolling the number of evaluable patients outlined in the table below.

| | | | |
|---|---|---|---|
| | | Maximum | |
| | | Number of | Number of |
| | Dose and | Patients | DLT-Evaluable |
| Tumor Type | Regimen | Enrolled | Subjects |

Cohort A - Dose Escalation (Proposed)
Compound 1 BID-QW

| | | | |
|---|---|---|---|
| Solid tumors | 50 mg BID-QW | 6 | Up to 40-50 |
| Solid tumors | 100 mg BID-QW | 6 | (including |
| Solid tumors | 125 mg BID-QW | 6 | backfill) |

| | | | |
|---|---|---|---|
| | | Number of | Number of |
| | Dose and | Patients | Evaluable |
| Tumor Type | Regimen | Enrolled | Subjects |

Cohort B - Dose Expansion
Compound 1 QW

| | | | |
|---|---|---|---|
| MAPKm/MAPKi-naïve pan | 250 mg QW | 16 + 15 | Up to 190 |
| tumor (−) CRC/NSCLC | | | |
| MAPKm/MAPKi-naïve pancreatic | 250 mg QW | 16 + 15 | |
| MAPKm/MAPKi-naïve NSCLC | 250 mg QW | 16 + 15 | |
| BRAFi-treated V600 NSCLC | 250 mg QW | 16 + 15 | |
| BRAFi-treated V600 melanoma | 250 mg QW | 16 + 15 | |
| KRAS-treated G12C NSCLC | 250 mg QW | 16 + 15 | |

Cohort C - Dose Expansion
Compound 1 RD BIW

| | | | |
|---|---|---|---|
| MAPKm/MAPKi-naïve pan | RD BID-QW | 16 + 15 | Up to 190 |
| tumor (−) CRC/NSCLC | | | |
| MAPKm/MAPKi-naïve pancreatic | RD BID-QW | 16 + 15 | |
| MAPKm/MAPKi-naïve NSCLC | RD BID-QW | 16 + 15 | |
| BRAFi-treated V600 NSCLC | RD BID-QW | 16 + 15 | |
| BRAFi-treated V600 melanoma | RD BID-QW | 16 + 15 | |
| KRAS-treated G12C NSCLC | RD BID-QW | 16 + 15 | |

Based on the clinical study described above, a favorable BID-QW dosing schedule was identified.

### Drug Product

Compound 1 drug product was supplied as an immediate-release oral dosage formulation in the form of 10 mg or 50 mg tablet for oral administration. The tablets contained 10 mg or 50 mg of Compound 1 with the inactive excipients hydroxypropyl cellulose, mannitol, microcrystalline cellulose, crospovidone, magnesium stearate, and Opadry White aesthetic coating.

Compound 1 was stored at room temperature (20 20-25□C [68-77□F]) with excursions permitted to 15 to 30□C (59-86□F) and was periodically tested and monitored for acceptable shelf life.

### Example 2

Both QW and QD regimens were evaluated in the First-in-Human (FIH) Phase 1 study. The data indicate that QW is preferred to QD for better efficacy and less frequent skin toxicities. The observed data in indicate that having an uninterrupted coverage over IC₉₀ for 1-2 days can improve cancer cell killing and maintaining C_{trough} below IC₅₀ can help normal cell recovery and can improve tolerability.

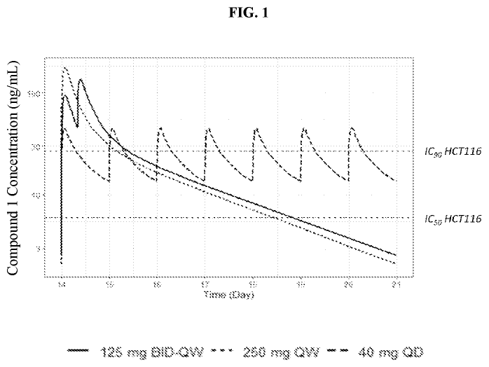

Based on the available Compound 1 human PK data, a population PK model was developed to characterize the PK characteristics. Using the developed PK model, simulations were performed to compare the PK performance of various dosing regimens (see FIG. 1). The simulations indicate that compared to QD dosing, QW and BID-QW regimens extended the duration above IC₉₀ while maintaining C_{trough} near or below IC₅₀ to give normal cells a treatment break. In addition, compared to QW, BID-QW is expected to have lower Cₘₐₓ and comparable C_{avg} and Cₘᵢₙ. With lower Cₘₐₓ, BID-QW regimen can further improve Cₘₐₓ-driven tolerability while maintaining comparable efficacy relative to a QW regimen.

1. A method of treating cancer in a subject in need thereof, the method comprising administering to the subject in need thereof:
compound 1 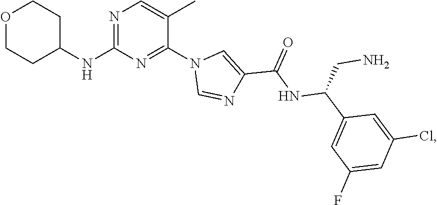 [SMILES: Cc1cnc(NC2CCOCC2)nc1-n1cnc(C(=O)N[C@H](CN)c2cc(F)cc(Cl)c2)c1]
or a pharmaceutically acceptable salt thereof;
wherein compound 1 is administered twice a day, once a week (BID-QW).

2. The method of claim 1, wherein the salt of compound 1, is the mandelic acid salt.

3. The method of claim 1 or 2, wherein compound 1, or a pharmaceutically acceptable salt thereof, is administered for at least one 28-day cycle.

4. The method of any one of claims 1-3, wherein compound 1, or a pharmaceutically acceptable salt thereof, is administered twice a day on day 1, day 8, day 15, and day 22 of a 28-day cycle.

5. The method of any one of claims 1-3, wherein compound 1, or a pharmaceutically acceptable salt thereof, is administered twice a day on day 1, day 8, day 15 of a 28-day cycle.

6. The method of any one of claims 1-5, wherein compound 1, or a pharmaceutically acceptable salt thereof, is not administered on days 2-7, days 9-14, days 16-21, and days 23-28 of a 28-day cycle.

7. The method of any one of claims 1-2, wherein compound 1, or a pharmaceutically acceptable salt thereof, is administered for at least one 35-day cycle.

8. The method of claim 7, wherein compound 1, or a pharmaceutically acceptable salt thereof, is administered twice a day on day 1, day 8, day 15, and day 22 of a 35-day cycle.

9. The method of any one of claims 7-8, wherein compound 1, or a pharmaceutically acceptable salt thereof, is not administered on days 2-7, days 9-14, days 16-21, days 23-28, and days 30-35 of a 28-day cycle.

10. The method of any one of claims 1-9, wherein compound 1, or a pharmaceutically acceptable salt thereof, is administered in an amount that is between about 25 mg and about 300 mg twice a day, once a week (BID-QW).

11. The method of any one of claims 1-10, wherein compound 1, or a pharmaceutically acceptable salt thereof, is administered in an amount that is between about 25 mg and about 250 mg twice a day, once a week (BID-QW).

12. The method of any one of claims 1-11, wherein compound 1, or a pharmaceutically acceptable salt thereof, is administered in an amount that is between about 25 mg and about 150 mg twice a day, once a week (BID-QW).

13. The method of any one of claims 1-11, wherein compound 1, or a pharmaceutically acceptable salt thereof, is administered in an amount that is about 25 mg, 50 mg, about 75 mg, about 100 mg, about 125 mg, about 150 mg, about 175 mg, about 200 mg, about 225 mg, or about 250 mg twice a day, once a week (BID-QW).

14. The method of any one of claims 1-13, wherein compound 1, or a pharmaceutically acceptable salt thereof, is administered in an amount that is about 25 mg, 50 mg, about 100 mg, about 125 mg, or about 150 mg twice a day, once a week (BID-QW).

15. The method of any one of claims 1-14, wherein the cancer comprises at least one cancer cell with one or more activating mutations in the MAPK pathway.

16. The method of any one of claims 1-14, wherein the cancer is a mitogen-activated protein kinase (MAPK) pathway driven cancer.

17. The method of claim 16, wherein the cancer is a BRAF-driven cancer, HRAS-driven cancer, or a NRAS-driven cancer.

18. The method of any one of claims 1-14, wherein the cancer comprises at least one cancer cell driven by deregulated ERK.

19. The method of any one of claims 1-15, wherein the cancer has at least one mutation in RAS, RAF, or MEK.

20. The method of any one of claims 1-19, wherein the subject has not previously received a MAPK pathway inhibitor (e.g., a KRAS inhibitor, a BRAF inhibitor, a MEK inhibitor, or an ERK inhibitor).

21. The method of any one of claims 1-20, wherein the cancer is colorectal cancer (CRC), pancreatic ductal adenocarcinoma (PDAC), cholangiocarcinoma cancer, appendiceal cancer, gastric cancer, esophageal cancer, non-small cell lung cancer (NSCLC), head and neck cancer, ovarian cancer, uterine cancer, or acute myeloid leukemia (AML).

22. The method of any one of claims 1-20, wherein the cancer is non-small cell lung cancer (NSCLC), melanoma, pancreatic cancer, or colorectal cancer (CRC).

23. The method of any one of claims 1-22, wherein the cancer is a MAPKm/MAPKi-naïve pan tumor, MAPKm/MAPKi-naïve pancreatic cancer, MAPKm/MAPKi-naïve NSCLC, BRAFi-treated V600 NSCLC, BRAFi-treated V600 melanoma, or KRAS-treated G12C NSCLC.

24. The method of any one of claims 1-23, wherein the subject in need thereof does not experience serious adverse events during the 28-day cycles.

25. The method of any one of claims 1-23, wherein the subject in need thereof does not experience Grade 3, 4, or 5 adverse events during the 28-day cycles.
